(19) United States
(12) Patent Application Publication
(10) Pub. No.: US 2026/0176686 A1
(43) Pub. Date: Jun. 25, 2026

(54) SINGLE-CELL TRANSCRIPTOME ANALYSIS OF HUMAN AMNION

(71) Applicant: California Institute of Technology, Pasadena, CA (US)

(72) Inventors: Magdalena D. Zernicka-Goetz, Pasadena, CA (US); Wenqi Hu, Pasadena, CA (US)

(21) Appl. No.: 19/425,783

(22) Filed: Dec. 18, 2025

Related U.S. Application Data
(60) Provisional application No. 63/736,049, filed on Dec. 19, 2024.

(51) Int. Cl. C12Q 1/6869; C12N 5/073; C12N 9/22; C12N 15/113; C12Q 1/6881

(52) U.S. Cl. CPC C12Q 1/6869; C12N 5/0605; C12N 9/222; C12N 15/113; C12Q 1/6881; C12N 2310/20; C12N 2501/20; C12N 2506/45

(57) ABSTRACT

Provided herein include methods, compositions, isolated cells, cell models, and kits suitable for use in profiling various cell types in amnion including amnion epithelial cells, amnion mesenchymal cells, fibroblasts, macrophages, as well as amnion stem cells including amnion epithelial stem cells and amnion mesenchymal stem cells. The present disclosure also discloses novel markers for various cell types in human amnion, including marker genes, transcription factors and signaling molecules associated with various stages of amniotic development. The methods, cell models, and gene signatures disclosed herein can be used for a variety of research, diagnostic, or therapeutic applications.

### RELATED APPLICATIONS

This application claims the benefit under 35 U.S.C. § 119 (e) of U.S. Provisional Patent Application Ser. No. 63/736,049, filed on Dec. 19, 2024, the content of this related application is incorporated herein by reference in its entirety for all purposes.

### STATEMENT REGARDING FEDERALLY SPONSORED R&D

This invention was made with government support under Grant No. HD104575 awarded by the National Institutes of Health. The government has certain rights in the invention.

### BACKGROUND

### Field

The present disclosure relates generally to the field of embryo development.

### Description of the Related Art

A comprehensive understanding of human amnion development remains limited. Single-cell RNA sequencing (scRNA-seq) of human pregastrulation embryos and of primate gastrulating embryos has provided transcriptional snapshots at specific stages of early development, offering preliminary insights into amnion development. Advances in stem cell technology, including the generation of stem cell-derived embryo-like models and stem cell-derived amnion-like cells, have further clarified the developmental pathway of amnion specification. RNA sequencing (RNA-seq) of amnion tissue collected from pregnant women at term has confirmed the presence of multiple cell types within the fully developed amnion, including fibroblasts, epithelial cells, immunocytes and various intermediate cell types. However, characterizing amnion from the early stages of human pregnancy remains challenging.

### SUMMARY

Provided herein includes an amnion cell model, comprising one or more cell types selected from amnion epithelial cells, amnion epithelial cells, fibroblasts, macrophages, amnion mesenchymal stem cells, and amnion epithelial stem cells, or comprising one or more cell types characterized by expression of amnion epithelial cell markers, amnion mesenchymal cell markers, fibroblast markers, macrophage markers, amnion mesenchymal stem cell markers, and/or amnion epithelial stem cell markers. Also provided herein includes an isolated amnion cell population, comprising cells selected from amnion epithelial cells, amnion epithelial cells, fibroblasts, macrophages, amnion mesenchymal stem cells, and amnion epithelial stem cells.

In some embodiments, the amnion cell model or the isolated amnion cell population comprises amnion epithelial cell, amnion mesenchymal cells, or both. In some embodiments, the amnion cell model or the isolated amnion cell population comprises amnion epithelial stem cells, amnion mesenchymal stem cells, or both. In some embodiments, the amnion epithelial cells are characterized by expression of GABRP, KRT18, IGFBP2, IGFBP5, NPY, FN1, ITGB6, VTCN1, CLDN6, STMN1, TYMS, MAD2L1, CDK1, ZWINT, TK1, CDH1, and a combination thereof, optionally, GABRP and KRT18.

In some embodiments, the amnion epithelial stem cells are characterized by expression of GABRP, KRT18, IGFBP2, VTCN1, CLDN6, STMN1, TYMS, MAD2L1, CDK1, ZWINT, TK1, CDH1, and a combination thereof. In some embodiments, the amnion mesenchymal cells are characterized by expression of MGP, DLK1, VIM, IGFL2, POSTN, COL8A2, COL15A1, COL6A1, COL5A1, ADAMTS9, or a combination thereof, optionally MGP and VIM. In some embodiments, the amnion mesenchymal stem cells are characterized by expression of STMN1, TYMS, MAD2L1, CDK1, MGP, DLK1, VIM, IGFL2, POSTN, PCLAF, CENPK, CENPM, ZWINT, TK1, or a combination thereof, optionally MGP, VIM, and PCLAF. In some embodiments, the amnion cells are obtained from human amnion cells, optionally from a first trimester human amnion tissue. In some embodiments, the amnion cells are obtained from in vitro generated amnion models. The amnion cells can be generated, e.g., by differentiation of pluripotent or multipotent stem cells. In some embodiments, the amnion cells correspond to human amnion cells in Carnegie stage (CS) 5, 6, 7, 8, 9, 10, 11, 12, 13, 14, 15, 16, 17, 18, 19, 20, 21, 22, or 23, optionally Carnegie stages (CS) 16, 17, 19, 22 and 23. In some embodiments, the amnion cell model or the isolated amnion cell population comprises amnion cells engineered to express or overexpress one or more amnion epithelial markers or one or more amnion mesenchymal markers. In some embodiments, the one or more amnion epithelial markers are selected from: GABRP, KRT18, IGFBP2, IGFBP5, NPY, FN1, ITGB6, VTCN1, CLDN6, STMN1, TYMS, MAD2L1, CDK1, ZWINT, TK1, CDH1, and a combination thereof, optionally, the one or more amnion epithelial markers comprise GABRP and KRT18, and the one or more amnion mesenchymal markers are selected from the group consisting of: MGP, DLK1, VIM, IGFL2, POSTN, COL8A2, COL15A1, COL6A1, COL5A1, ADAMTS9, or a combination thereof, optionally the one or more amnion mesenchymal markers comprise MGP and VIM. In some embodiments, the engineered amnion cells are enriched in transcription factors GATA6, TWIST1 and ZEB1 or enriched in transcription factors TFAP2A and TFAP2B.

Disclosed herein includes a method of detecting perturbation-induced changes in amnion cells, comprising: introducing a perturbation to an amnion cell model or an isolated amnion cell population of any one of claims 1-14; and determining a perturbation-induced change in one or more gene signatures or pathways of the amnion cell model or the isolated amnion cell population, and evaluating the effect of the perturbation on the amnion cell model or the isolated amnion cell population by detecting expression of one or more amnion epithelial cell markers, amnion mesenchymal cell markers, fibroblast markers, macrophage markers, amnion mesenchymal stem cell markers, and/or amnion epithelial stem cell markers.

The perturbation can be a physical condition or a chemical condition. In some embodiments, the perturbation comprises a genetic perturbation, a drug candidate, an absence or presence of a test agent, an increase or decrease of temperature, light, pressure, pH value, or a combination thereof. In some embodiments, the perturbation comprises an agent selected from cytokines, a transcription factors, signaling molecules, or a combination thereof.

In some embodiments, introducing the perturbation to the amnion cell model or the isolated amnion cell population comprises contacting the amnion cell model or the isolated amnion cell population with the perturbation in a culture medium or culturing the amnion cell model or the isolated amnion cell population in a culture medium in the presence of the perturbation. In some embodiments, introducing the perturbation to the amnion cell model or the isolated amnion cell population comprises performing RNA interference or CRISPR-Cas gene editing. In some embodiments, determining the perturbation-induced change in the one or more gene signatures or pathways comprises determining the expression level of the one or more amnion epithelial cell markers, amnion mesenchymal cell markers, or both. In some embodiments, determining the perturbation-induced change in the one or more gene signatures or pathways comprises comparing the one or more gene signatures or pathways of the amnion cell model or isolated amnion cell population in the presence of the perturbation with one or more gene signatures or pathways of the amnion cell model or isolated amnion cell population in the absence of the perturbation. In some embodiments, determining the perturbation-induced change in the one or more gene signatures or pathways comprises performing single cell sequencing. In some embodiments, the amnion epithelial cell markers comprise GABRP and KRT18, and the amnion mesenchymal cell markers comprise MGP and VIM.

Disclosed herein includes a method of identifying amnion epithelia cells, comprising: detecting in a cell population one or more genes or gene expression products selected from the group consisting of: GABRP, KRT18, IGFBP2, IGFBP5, NPY, FN1, ITGB6, VTCN1, CLDN6, STMN1, TYMS, MAD2L1, CDK1, ZWINT, TK1, CDH1, and a combination thereof, optionally, GABRP and KRT18. Disclosed herein includes a method of identifying amnion epithelia stem cells, comprising: detecting in a cell population one or more genes or gene expression products selected from the group consisting of: GABRP, KRT18, IGFBP2, VTCN1, CLDN6, STMN1, TYMS, MAD2L1, CDK1, ZWINT, TK1, CDH1, and a combination thereof. Also disclosed herein includes a method of identifying amnion mesenchymal cells, comprising: detecting in a cell population one or more genes or gene expression products selected from MGP, DLK1, VIM, IGFL2, POSTN, COL8A2, COL15A1, COL6A1, COL5A1, ADAMTS9, or a combination thereof, optionally MGP and VIM. Also disclosed herein includes a method of identifying amnion mesenchymal stem cells, comprising: detecting in a cell population one or more genes or gene expression products selected from STMN1, TYMS, MAD2L1, CDK1, MGP, DLK1, VIM, IGFL2, POSTN, PCLAF, CENPK, CENPM, ZWINT, TK1, or a combination thereof, optionally MGP, VIM, and PCLAF. In some embodiments, the methods further comprises obtaining a single cell gene expression profile for the one or more cells, optionally by performing single cell sequencing. In some embodiments, the cell population is obtained from a human amnion sample, from an in vitro generated amnion models, or generated by differentiation of pluripotent or multipotent stem cells. In some embodiments, the human amnion sample is a first trimer human amnion tissue.

Disclosed herein includes a method of differentiating stem cells into amnion cells, comprising culturing a population of induced pluripotent stem (iPS) cells in the presence of one or more agents capable of modulating expression of one or more amnion marker genes in a culture medium under a condition allowing the iPS cells to differentiate into amnion cells, wherein the one or more amnion marker genes is selected from the group consisting of: GABRP, KRT18, IGFBP2, IGFBP5, NPY, FN1, ITGB6, VTCN1, CLDN6, STMN1, TYMS, MAD2L1, CDK1, ZWINT, TK1, CDH1, MGP, DLK1, VIM, IGFL2, POSTN, COL8A2, COL15A1, COL6A1, COL5A1, ADAMTS9, PCLAF, CENPK, CENPM, COL6A1, MALATI, MRC1, CD36, SRGN, FOLR2, PTPRC, or a combination thereof. In some embodiments, the one or more agents comprises one or more cytokines, growth factors, hormones, transcription factors, and/or signaling molecules. In some embodiments, the signaling molecules are selected from the group consisting of: BMP10, BMP3, BTC, MIF, IGF2, PDGFA, AREG, PDGFC, WNT2B, WNT10A, WNT1, WNT10B, WNT5A, WNT7A, WNT4, WNT6, WNT3, BMP7, NOG, PDGFD, WNT9B, INHBB, WNT7B, EGF, MSTN, GDF11, WNT3A, NDP, WNT8B, BMP6, TGFA, GDF9, CHRD, GREM2, MDK, BMP4, WNT2, TGFB2, TGFB3, BMP5, HGF, FST, WNT16, LEFTY1, WNT9A, TGFB1, BMP2, LEFTY2, SPP1, IGF1, HBEGF, PDGFB, WNT11, GDF5, GDF15, GREM1, EPGN, INHBA, and a combination thereof. In some embodiments, the transcription factors are selected from the group consisting of: POU5F1, HAND1, ISL1, ID3, MSX1, EPAS1, ID4, PRRX1, EN1, HOXB6, ID1, DLX5, ID2, MYBL2, TCF4, HOXA10, PPARG, MSX2, TBX3, HES1, TFAP2B, TFAP2A, IRX1, IRX2, MEIS2, GATA6, HAND2, LMCD1, PITX1, PITX2, SOX6, FOSL2, TWIST1, LEF1, ZEB1, TBX2, and a combination thereof. In some embodiments, the one or more agents comprises BMP4. In some embodiments, the method comprises measuring the expression level of the one or more amnion marker genes comprising GABRP, KRT18, MGP, and VIM.

Disclosed herein includes a method of treating a disease or disorder, comprising: administrating an effective amount of a population of amnion epithelial cells, amnion mesenchymal cells, or both to a subject in need, thereby providing an immunomodulatory or regenerative therapeutic effect in the subject. The method can further comprises quantifying expression and/or abundance of immunosuppressive factors of migration inhibitory factor (MIF), SPP1 and/or SPP1 receptor CD44 of the amnion cell population. In some embodiments, the amnion cell population is provided in an amnion cell sheet. Disclosed herein includes a method of elucidating the role of a gene in amniotic development, comprising: obtaining a pluripotent cell where the gene has been modified or knocked out, culturing the pluripotent cell in a culture medium under a condition allowing the pluripotent cell to differentiate into an embryonic structure, and measuring expression level of one or more amnion gene markers.

Also disclosed herein includes a kit comprising primers targeting at least 3, 4, 5, 6, 7 or 8 of the marker genes of GABRP, KRT18, IGFBP2, IGFBP5, NPY, FN1, ITGB6, VTCN1, CLDN6, STMN1, TYMS, MAD2L1, CDK1, ZWINT, TK1, CDH1, MGP, DLK1, VIM, IGFL2, POSTN, COL8A2, COL15A1, COL6A1, COL5A1, ADAMTS9, PCLAF, CENPK, CENPM, COL6A1, MALATI, MRC1, CD36, SRGN, FOLR2, or PTPRC. The kit can comprise primers targeting GABRP, IGFBP3, KRT18, MGP, VIM, and CD36.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts non-limiting exemplary data related to scRNA-seq analysis of human amnion during the first trimester. Panel a, A diagram illustrating the development of the human amnion. Trophectoderm is marked in grey, amnion in orange, extra-embryonic mesoderm in red, hypoblast or yolk sac endoderm in green, epiblast/embryo body in burgundy or pink and umbilical cord in purple. Panel b, Images of human embryos representing different stages. CS16-CS19 were the collected sample, and CS22 is a representative image taken from the same centre. Arrows point to the amniotic membrane, and triangles mark the yolk sac. Scale bar, 0.5 cm. Panel c, UMAP displaying the identified cell types within the analysed samples. Panel d, A bubble plot showing selected top marker gene expression across cell types. Panel e, Immunofluorescence (IF) staining of amnion sections, with protein markers labelled at the top of the image and amnion stages labelled at the bottom. Different fluorescent markers highlight the localization of the proteins in the tissue. The region inside the white box is magnified on the right. White arrows indicate double-positive cells. Scale bars, 50 μm. CD45-VIM co-staining is representative of four independent experiments that yielded similar results. CD45-E-Cadherin co-staining is representative of two independent experiments. VIM-E-Cadherin and N-Cadherin-KRT18 are representative of one experiment. scRNA-seq analyses depicted in this figure are generated from human amnion samples of the following developmental stages: CS16 (n=1), CS17 (n=1), CS19 (n=1) and CS22 (n=1).

FIGS. 2A-2G depict non-limiting exemplary data related to cell subtypes and lineage trajectories in human amnion. FIG. 2A displays a non-limiting exemplary a 3D UMAP representation showing different cell subtypes in the amnion. FIG. 2B displays non-limiting exemplary violin plots showing marker gene expression across subtypes. FIG. 2C displays non-limiting exemplary images showing in the top: immunostaining of GABRP and SOX2 in the CS16 amnion section, with triangles marking the ectodermal cells. Representative image from two independent experiments. Bottom: immunostaining of E-Cadherin and TUBB3 in the CS19 amnion section. Scale bars, 50 μm. Representative image from four independent experiments. FIG. 2D displays a non-limiting exemplary graph showing RNA velocity analysis indicating development tendencies of epithelial and mesenchymal cells, based on the integrated analysis of four independent biological samples. FIG. 2E displays non-limiting exemplary pseudotime and trajectory plots showing the epithelial-macrophage trajectory (top) and mesenchymal trajectory (bottom). FIG. 2F displays non-limiting exemplary graphs showing cell subtypes arranged along the Destiny pseudotime in two trajectories: epithelial-macrophage lineage (left) and mesenchymal lineage (right). FIG. 2G displays non-limiting exemplary graphs showing the expression of selected differentially expressed genes (DEGs) during lineage progression in amnion: macrophage (top), epithelial (middle) and mesenchymal (bottom). scRNA-seq analyses depicted in this figure are generated from human amnion samples of the following developmental stages: CS16 (n=1), CS17 (n=1), CS19 (n=1) and CS22 (n=1).

FIGS. 3A-E depict non-limiting exemplary data related to cellular communication and secretion patterns within the amnion. FIG. 3A displays a non-limiting exemplary graph showing that the total number of inferred signaling interactions among different cell types. FIG. 3B displays a non-limiting exemplary graph showing that overall interaction strength representing the cumulative communication probability between cell types. FIG. 3C displays a non-limiting exemplary graph showing cell roles in secreting and receiving signals. FIG. 3D displays a non-limiting exemplary graph showing classification of cells into three distinct secretion patterns based on gene expression profiles. FIG. 3E displays a non-limiting exemplary heatmap showing the expression of ligands across different cell types. scRNA-seq analyses depicted in this figure are generated from human amnion samples of the following developmental stages: CS16 (n=1), CS17 (n=1), CS19 (n=1) and CS22 (n=1).

FIGS. 4A-4I depict non-limiting exemplary data related to key signaling pathways in distinct amnion cell patterns. FIGS. 4A-4C display non-limiting exemplary chord diagrams showing ligand-receptor interactions in epithelial (a), mesenchymal (b) and macrophage (c) patterns. Distinct cell types are represented by different colours. FIG. 4D displays a non-limiting exemplary heatmap showing interactions in the BMP signaling pathway. Commun prob., communication probability. FIG. 4E displays a non-limiting exemplary bubble plot showing the significant interactions in the BMP signaling pathway. FIGS. 4F-4I display non-limiting exemplary heatmaps showing interactions in PDGF (f), IL6 (g), TGF-β (h) and SPP1 (i) signaling pathways. scRNA-seq analyses depicted in this figure are generated from human amnion samples of the following developmental stages: CS16 (n=1), CS17 (n=1), CS19 (n=1) and CS22 (n=1).

FIGS. 5A-5G depict non-limiting exemplary results from combined analysis of amnion data from human, monkey and in vitro stem cell-derived embryo models. FIG. 5A displays a non-limiting exemplary combined UMAP of amnion data from human, monkey and in vitro stem cell-derived models. NNE, non-neural ectoderm; AM, amnion; ExE.Meso, extra-embryonic mesoderm; ECT, ectoderm; EPI, epiblast; Mes, mesenchyme; SE1, surface ectoderm1; SE2, surface ectoderm2; VE, visceral endoderm; ESC, embryonic stem cell; AMLC, amnion-like cell; MeLC, mesoderm-like cell; PGCLC, primordial germ cell-like cell. Data were merged from the scRNA-seq data generated in this study and five published datasets. FIG. 5B displays a non-limiting exemplary diffusion map illustrating the distribution of various amnion and amnion-like cell populations based on the integrated dataset comprising our data and five published datasets. Arrows indicate the developmental process. AME-E, amnion early like cell; AME-L, amnion late-like cell; AP3, hPS cells that were primed for 3 days; AP8, hPS cells that were primed for 8 days. FIG. 5C displays non-limiting exemplary pseudotime plots of amnion and amnion-like cells derived from this study and five published datasets, with dataset origins indicated on the side. Pseudotime_dm; pseudotime computed using diffusion maps (dm). FIG. 5D displays a non-limiting exemplary heatmap showing the expression of each gene module across amnion and amnion-like cells from our data and five published datasets. Gene module numbers are shown on the left. Specific markers and transcription factors (TFs) are shown on the right. FIGS. 5E-5G displays non-limiting exemplary graphs showing GO enrichment analysis of early amnion (e), AMCs (f) and AECs (g). Data sources include previously published datasets from human CS7, monkey CS8-11, and three sets of in vitro-derived amnion-like cells.

FIG. 6 depicts non-limiting exemplary data related to quality control of single-cell RNA-seq data. Panel (a): UMAP visualization depicting Seurat-defined clusters using pre-QC data. Panel (b): Overlay of Souporcell-defined genotypes on the pre-QC UMAP. Cells clustering by the same genotype were regarded as maternal cells. Panel (c): Distribution of cell type scores on the pre-QC UMAP. Panel (d) Histogram presenting the number of cells across different scoring categories. scRNA-seq analyses depicted in this figure are generated from human amnion samples of the following developmental stages: CS16 (n=1), CS17 (n=1), CS19 (n=1), CS22 (n=1).

FIG. 7 depicts non-limiting exemplary data related to Seurat clusters and marker genes on UMAP. Panel (a): UMAP showing Seurat clusters after quality control, panel (b): Marker genes of epithelial, mesenchymal, and progenitor cells were shown on UMAP. scRNA-seq analyses depicted in this figure are generated from human amnion samples of the following developmental stages: CS16 (n=1), CS17 (n=1), CS19 (n=1), CS22 (n=1).

FIG. 8 depicts non-limiting exemplary data related to immunostainings of amnion sections in CS19 and CS23. (a) Immunostaining of CD45 and VIM in the human amnion section. left panel: CS19; right panel: CS23. Scale bar=50 μm. Representative image from 2 independent experiments. (b) Immunostaining of CD45 and VIM in the human amnion section. top panel: CS19; bottom panel: CS23, Scale bar=50 μm. Representative image from 2 independent experiments. (c) Immunostaining of CD45 and E-Cadherin in the human amnion section. top panel: CS19; bottom panel: CS23, Scale bar=50 μm. Representative image from 1 independent experiments. (d) Immunostaining of E-Cadherin and VIM in the human CS23 amnion section. Scale bar=50 μm. Representative image from 1 independent experiment. (e) Immunostaining of N-Cadherin and KRT18 in the human CS23 amnion section. Scale bar=50 μm. Representative image from 1 independent experiment.

FIG. 9 depicts non-limiting exemplary data related to cell subtype identification in human amnion. Panel (a): Seurat clusters on 3D UMAP. Panel (b): Heatmap of the top 50 epithelial (top) and mesenchymal marker genes (bottom) across cell subtypes. Panel (c): SOX2-positive cells were shown on UMAP. Panel (d): Bar plot showing the expression of neural-related genes in 9, 16-18 and 22 weeks of human amnion samples (bulk data from Roost, 2015). The samples at 9 weeks and 22 weeks each have two biological replicates, while the samples from 16-18 weeks have three biological replicates. Each data point represents an individual sample, and bars indicate the mean expression value. Panel €: Umap showing the expression of neural-related genes in amnion cells. Panel (f): Umap showing the Co-expression of SOX2 (yellow) and neural markers (blue). Double-positive cells are colored in purple. Cell proportions are list on the legend. Panel (g): Immunostaining of SOX2 and TUBB3 in the human CS19 amnion section. Scale bar=50 μm. White frame region is magnified and shown on the right. Top panel shows TUBB3 positive cells, bottom panel shows TUBB3, SOX2 double-positive cells. Representative image from 4 independent experiments, scRNA-seq analyses depicted in panels a, b and c are generated from human amnion samples of the following developmental stages: CS16 (n=1), CS17 (n=1), CS19 (n=1), CS22 (n=1). Analyses depicted in panels e and f are based on amnion cells from monkey CS8-11 dataset.

FIG. 10 depicts non-limiting exemplary data related to the expression of EMT-related TFs. Panel (a) Violin plots showing the expression of EMT-related TFs across different cell subtypes. Panel (b) SNAI1-positive cells were shown on UMAP, with an enlarged section on the right. scRNA-seq analyses depicted in this figure are generated from human amnion samples of the following developmental stages: CS16 (n=1), CS17 (n=1), CS19 (n=1), CS22 (n=1).

FIG. 11 depicts non-limiting exemplary data related to cell-cell communication network between any two cell groups. Panel (a) Circle plots showing cell-cell interactions. Line weights indicating the total interaction strength. scRNA-seq analyses depicted in this figure are generated from human amnion samples of the following developmental stages: CS16 (n=1), CS17 (n=1), CS19 (n=1), CS22 (n=1).

FIG. 12 depicts non-limiting exemplary data related to ligand/receptor interactions and gene expression. Panel (a) Circle plots depicting BMP ligand-receptor interactions. Upper panel: BMP4; Lower panel: BMP7. Panel (b) Violin plots showing the expression of BMP ligands and their receptors across different cell types. Panels (c, e) Heatmaps displaying the cell-cell interactions in the MDK (c) and WNT (e) signaling pathways. Panels (d, f) Bubble plots showing MDK (d) and WNT (f) ligand-receptor interactions across different cell types. Inter-cells, short for intermediate cells. scRNA-seq analyses depicted in this figure are generated from human amnion samples of the following developmental stages: CS16 (n=1), CS17 (n=1), CS19 (n=1), CS22 (n=1).

FIG. 13 depicts non-limiting exemplary data related to BMP4 induction and immunosuppressive factors expression in amnion. Panel (a) In vitro stem cell experiment design (left) and qPCR results (right) of cytokines-treated iPSCs. Relative gene expression was calculated using the ΔΔCt method, with GAPDH as the internal control. Each data point represents an individual sample, and bars indicate the mean expression value. The untreated medium was used as the control group, while groups treated with BMP4, RA, and MDK were the experimental groups. Each group, except for the BMP4-treated group, has two biological replicates. Panels (b, c) Circle plots showing MIF (b) and SPP1 (c) interactions across different cell types. Analyses are generated from human amnion samples of the following developmental stages: CS16 (n=1), CS17 (n=1), CS19 (n=1), CS22 (n=1). Panel (d) Immunostaining showing the expression of SPP1, MIF and CD44 in the CS16 amnion section, Scale bar=50 μm. Representative image from 3 independent experiments.

FIG. 14 depicts non-limiting exemplary data related to single-cell RNA-seq datasets of amnion and amnion models. Panel (a) UMAP of single-cell RNA-seq data from the human CS7 amnion. Panel (b) UMAP of single-cell RNA-seq data from the monkey CS8-11 amnion. Panels (c-e) UMAPs of single-cell RNA-seq data from in vitro amnion models.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include amnion cell models and cells, biomarkers and/or gene signatures identified in various amnion cell types, and related methods, compositions and kits for therapeutic, diagnostic, or screening uses.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 0.5 would include 0.45 and 0.55, about 10 would include 9 to 11, about 1000 would include 900 to 1100.

The term “stem cell,” as used herein, refers to a cell that is capable of differentiating into one or more differentiated cell types. Stem cells may be totipotent. Stem cells may be pluripotent cells. Totipotent stem cells typically have the capacity to develop into any cell type. Totipotent stem cells are usually embryonic in origin. The term “progenitor cell,” as used herein, refers to a cell that is committed to a particular cell lineage and which gives rise to a particular limited range of differentiated cell types by a series of cell divisions. An example of a progenitor cell would be a myoblast, which is capable of differentiation to only one type of cell, but is itself not fully mature or fully differentiated.

The term “pluripotent stem cell” (PSC) as used herein refers to a stem cell permitting in vitro culture and having the potential for differentiating into all cells, but the placenta. The pluripotent stem cell has the potential to differentiate into any of the three germ layers: endoderm (which forms structures such as the gastrointestinal tract and the respiratory system), mesoderm (which forms structures such as the musculoskeletal system, the vascular system and the urogenital system), or ectoderm (which forms epidermal tissues and the nervous system). A PSC may be obtained from a fertilized egg, clone embryo, reproductive stem cell, or stem cell in tissue. Also included are cells having differentiation pluripotency similar to that of embryonic stem cells, conferred artificially by transferring several different genes to a somatic cell (also referred to as induced pluripotent stem cells or iPS cells). Induced pluripotent stem cells may be derived from any suitable source (e.g. hair follicles, skin cells, fibroblasts, etc.). Pluripotent stem cells can be prepared by known methods in the art. Non-limiting examples of said stem cells include embryonic stem cells of a mammal or the like established by culturing a pre-implantation early embryo, embryonic stem cells established by culturing an early embryo prepared by nuclear-transplanting the nucleus of a somatic cell, induced pluripotent stem cells (iPS cells) established by transferring several different transcriptional factors to a somatic cell, and pluripotent stem cells prepared by modifying a gene on a chromosome of embryonic stem cells or iPS cells using a gene engineering technique. More specifically, embryonic stem cells include embryonic stem cells established from an inner cell mass that constitutes an early embryo, ES cells established from a primordial germ cell, cells isolated from a cell population possessing the pluripotency of pre-implantation early embryos (for example, primordial ectoderm), and cells obtained by culturing these cells.

As used herein, the term “differentiation” can refer to the process by which an unspecialized (“uncommitted”) or less specialized cell acquires the features of a specialized cell such as, for example, a neuronal cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. As used herein, a “lineage-specific marker” can refer to a characteristic specifically associated with the phenotype of cells of a lineage of interest and can be used to assess the differentiation of an uncommitted cell to the lineage of interest.

As used herein, “markers”, “lineage markers” or, “lineage-specific markers” can refer to nucleic acid or polypeptide molecules that are differentially expressed in a cell of interest. Differential expression can mean an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art. In some embodiments, a marker can be enriched. The term “enriched”, as used herein, shall have its ordinary meaning, and can also refer to a statistically significant increase in levels of a gene product (e.g., mRNA and/or protein) in one condition as compared to another condition (e.g., in one cell layer as compared to another cell layer).

The term, “concentration” as used herein shall have its ordinary meaning, and can also refer to (a) mass concentration, molar concentration, volume concentration, mass fraction, molar fraction or volume fraction, or (b) a ratio of the mass or volume of one component in a mixture or solution to the mass or volume of another component in the mixture or solution (e.g., ng/ml). In some embodiments, the concentration can refer to fraction of activity units per volume (e.g., U/ml).

The term “analogue” as used herein refers to a compound which may be structurally related to the relevant molecule. The term “agonist” as used herein can refer to a compound which might not be structurally related to the relevant molecule. For example, an agonist may activate the relevant receptor by altering the conformation of the receptor. Nevertheless, in both cases the terms are used in this specification to refer to compounds or molecules which can mimic, reproduce or otherwise generally substitute for the specific biological activity of the relevant molecule.

As used herein the phrase “culture medium” or “media” refers to a liquid substance used to support the growth and development of stem cells and of an embryo. The culture medium used according to some embodiments of the invention can be a water-based medium which includes a combination of substances such as salts, nutrients, minerals, vitamins, amino acids, nucleic acids, and/or proteins such as cytokines, growth factors and hormones needed for cell growth and embryo development.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

### Amnion

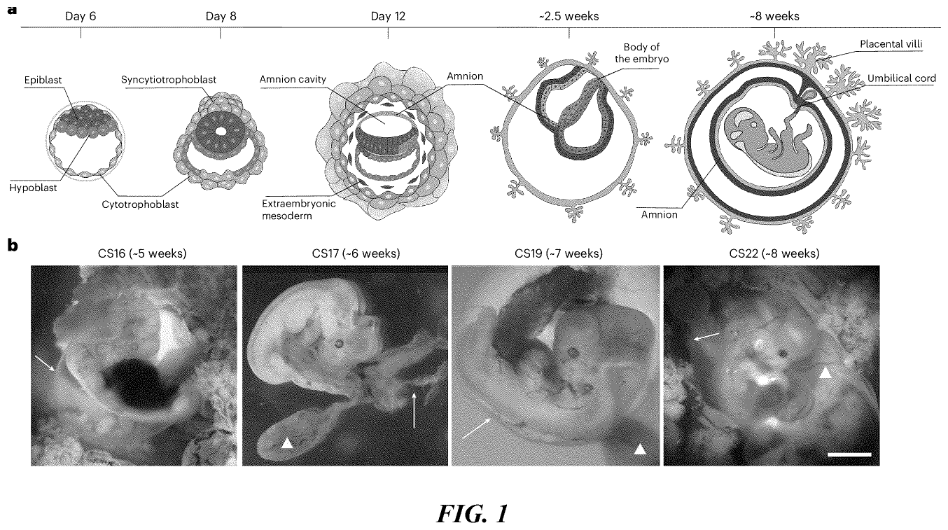

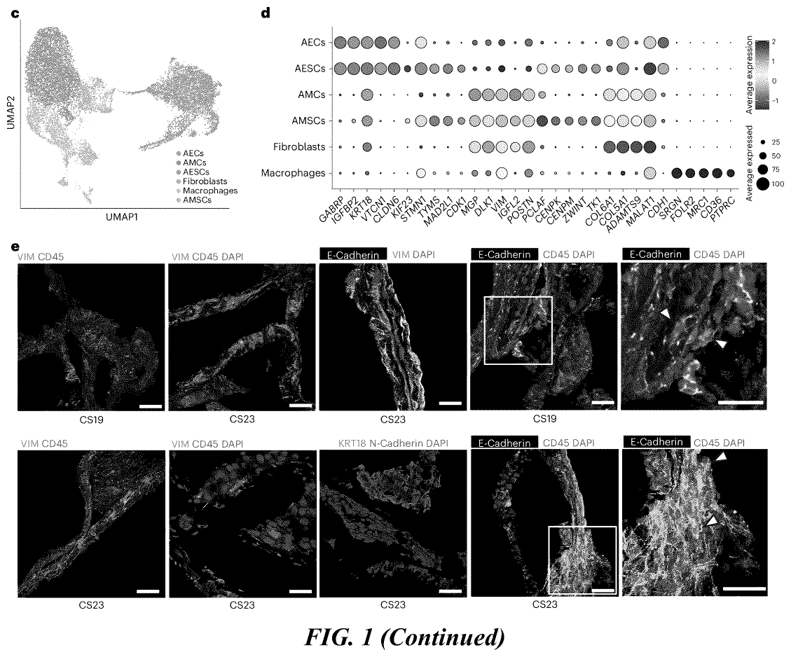

The amnion is an extra-embryonic structure essential for the development of reptilian, avian and mammalian embryos as it encases the embryo, providing both mechanical and biochemical support. In humans, the amnion originates from a subset of pluripotent epiblast cells specified soon after implantation. Before implantation, the blastocyst consists of an outer extra-embryonic trophoblast layer and the inner cell mass that will differentiate into epiblast and extra-embryonic hypoblast by days 6-7 post-fertilization. Upon implantation, epiblast cells polarize to form a rosette structure that undergoes lumenogenesis, creating the amniotic cavity as cells exit naive pluripotency. Epiblast cells in contact with the hypoblast form the epiblast disc, which will develop into the embryo proper, while those in contact with the trophectoderm will form the amnion (FIG. 1, panel a). The amnion not only physically protects the embryo but also secretes essential hormones and cytokines that support embryonic development.

The human amnion is composed of two primary cell types-epithelial cells and mesenchymal cells-separated by a thick basement membrane. Amniotic epithelial cells, which line the amniotic cavity, are responsible for producing the amniotic fluid, whereas the amniotic mesenchymal cells, embedded within the extracellular matrix, contribute to the structural scaffold of the avascular foetal membranes. These membranes define the intrauterine cavity and protect the foetus during gestation.

The amnion undergoes extensive growth, repair and remodelling throughout pregnancy to align with embryonic development. These processes are closely associated with epithelial-to-mesenchymal transitions (EMT) and mesenchymal-to-epithelial transitions (MET). In addition, EMT in the amnion has been reported to influence the immune properties of amniotic epithelial cells, often associated with localized inflammation and facilitating tissue remodelling.

Beyond its fundamental role in pregnancy, the human amnion serves as a valuable source of stem cells with multilineage differentiation potential. The stem cells derived from amnion can be utilized for cell-based therapies and regenerative medicine applications. The unique properties of the amnion, including low immunogenicity, anti-inflammatory and antimicrobial properties, make it an attractive candidate for various therapeutic applications, such as wound healing, treatment of ocular surface disorders and tissue engineering.

Despite growing interest, a comprehensive understanding of human amnion development remains limited. Single-cell RNA sequencing (scRNA-seq) of human pregastrulation embryos and of primate gastrulating embryos has provided transcriptional snapshots at specific stages of early development, offering preliminary insights into amnion development. Advances in stem cell technology, including the generation of stem cell-derived embryo-like models and stem cell-derived amnion-like cells, have further clarified the developmental pathway of amnion specification. RNA sequencing (RNA-seq) of amnion tissue collected from pregnant women at term has confirmed the presence of multiple cell types within the fully developed amnion, including fibroblasts, epithelial cells, immunocytes and various intermediate cell types. However, characterizing amnion from the early stages of human pregnancy remains challenging.

There are provided, in some embodiments, methods, compositions, cell models, and kits suitable for use in profiling various cell types in amnion including amnion epithelial cells, amnion mesenchymal cells, fibroblasts, macrophages, as well as amnion stem cells including amnion epithelial stem cells and amnion mesenchymal stem cells. In some embodiments, the present disclosure uses scRNA-seq to profile various cell types and subtypes present in human amnion during the first trimester of human pregnancy to gain insight into their interactions and potential functional contributions. The present disclosure provide a detailed view of amnion cellular compositions and interactions. Provided herein also include biomarkers and/or gene signatures identified in various cell types and subtypes in amnion and uses thereof. The methods and biomarkers/gene signatures disclosed herein can be used for any therapeutic, diagnostic or screening methods described herein, including, for example, detecting amnion cell types expressing a specific gene signature as well as detecting perturbation-induced change in amnion cells, e.g., for screening test agents. Other aspects, features, and advantages of the example embodiments will become apparent to those having ordinary skill in the art.

Some of the methods and compositions disclosed herein are also disclosed in Hu W. et al. “Atlas of amnion development during the first trimester of human pregnancy” (Nat Cell Biol 27, 1175-1185 (2025). https://doi.org/10.1038/s41556-025-01696-9), the content of which is incorporated herein by reference in its entirety.

### Stem Cells and Embryo Development

Mammalian embryogenesis is the process of cell division and cellular differentiation during early prenatal development which leads to the development of a mammalian embryo. While mammalian embryogenesis has some common features across all species, it will be appreciated that different mammalian species develop in different ways and at different rates. In general, though, the fertilized egg undergoes a number of cleavage steps (passing through two cell, four cell and eight cell stages) before undergoing compaction to form a solid ball of cells called a morula, in which the cells continue to divide. Ultimately the internal cells of the morula give rise to the inner cell mass and the outer cells to the trophectoderm. The morula in turn develops into the blastocyst, which is surrounded by trophectoderm and contains a fluid-filled vesicle, with the inner cell mass at one end.

The term “embryo” as used herein refers to a mammalian organism from the single cell stage. A developmental stage of an embryo can be defined by the development of specific structures and can be used to define equivalent stages in development of other species. In some embodiments, a developmental stage of an embryo can be defined according to “Carnegie stages”, which is a standardized system used to provide a unified developmental chronology of the vertebrate embryo.

In some embodiments, the embryo described herein is generated from culturing in vitro under appropriate conditions and resembles a natural embryo produced in vivo of a corresponding stage, such as having similar morphology, length, weight, cell type compositions and expression of developmental marker genes.

A developmental stage of an embryo can be defined by the development of specific structures and can be used to define equivalent stages in development of other species. In some embodiments, a developmental stage of an embryo can be defined according to “Carnegie stages”, which is a standardized system used to provide a unified developmental chronology of the vertebrate embryo. Table 1 below provides the characteristics of different Carnegie Stages in human (Life (Basel), 2023 Apr. 26; 13 (5): 1084).

TABLE 1
Carnegie Stages of Development

| | Days | | |
|---|---|---|---|
| | since | | |
| Carnegie | ovulation | Size | |
| stage | (approx.) | (mm) | Characteristics |
| 1 | 1 | 0.125 | fertilization; unicellular |
| 2 | 1.5-3 | 0.15 | More than one cell presentNo |
| | | | blastocystic cavity present |
| 3 | 4 | 0.15 | Blastocyst |
| 4 | 5-6 | 0.15 | Zona pellucida dissolved |
| | | | Blastocyst attachment to uterine |
| | | | epithelium |
| 5 | 7-12 | 0.15 | Solid trophoblast |
| | | | Trophoblastic lacunae |
| | | | Primary umbilical vesicle |
| | | | Mesoblastic crests |
| | | | Lacunar vascular circle |
| 6 | 13 | 0.2 | Chorionic villi |
| | | | Primitive streak |
| | | | Secondary umbilical vesicle |
| | | | Cloacal membrane |
| 7 | 16 | 0.4 | Cranial prolongation primitive streak |
| | | | (notoch. process) |
| | | | Primitive node |
| | | | Secondary villi |
| | | | Cloacal membrane |
| | | | Allantoic diverticulum |
| 8 | 18 | 1.25 | Primitive node |
| | | | Notochordal process |
| | | | Prechordal plate |
| | | | Primitive pit |
| | | | Notochordal canal |
| 9 | 20 | 2 | Head fold |
| | | | Somite pairs |
| 10 | 22 | 2.25 | Neural tube closing |
| | | | Looped heart tube |
| 11 | 24 | 3.5 | Rostral neuropore closing |
| | | | Otic placodes |
| | | | Optic vesicles |
| | | | 1st and 2nd phar. arches |
| | | | Meson. duct and tubules |
| | | | Sinus venosus |
| 12 | 26 | 4 | Rostral neuropore closed |
| | | | Caudal neuropore closing; |
| | | | Upper limb buds |
| | | | 3rd pharyngeal arch |
| | | | Otic pits |
| | | | Lung bud |
| | | | Interventricular septum formation |
| 13 | 28 | 5 | Lower limb buds appear as bulges |
| | | | Caudal neuropore closed |
| | | | Lens placodes |
| | | | Otic vesicles |
| | | | Left and right lung buds discernable |
| | | | Septum primum and foramen primum |
| 14 | 32 | 6 | Longer upper limbs |
| | | | Lower limbs clearly visible |
| | | | Nasal pits |
| | | | Optic cups |
| 15 | 33 | 8 | Handplate |
| | | | Lower limbs elongate |
| | | | Future cerebral hemispheres distinct |
| | | | Foramen secundum in the heart |
| 16 | 37 | 9.5 | Slight rotation upper limbs |
| | | | Footplate |
| | | | Pigment in the retina |
| 17 | 41 | 12.5 | Digital rays in hand plate |
| | | | Slight rotation of lower limbs |
| | | | Cerebral vesicles clearly visible |
| | | | Semilunar cusps visible in the heart |
| | | | Foramen primum obliterated |
| 18 | 44 | 15 | Longer and straighter trunk, toe rays |
| | | | Scalloping hand plate, start digits |
| | | | 4th ventricle larger than lateral ventr. |
| | | | Elbow region visible |
| | | | Membran. region interventr. septum |
| | | | Septum secundum |
| 19 | 47.5 | 18.5 | Elongation and straightening of trunk |
| | | | Upper limbs slightly bent in elbow |
| | | | Limbs extend ventrally |
| | | | Hands far apart, short fingers |
| | | | Midgut herniation |
| 20 | 50.5 | 22 | Longer upper limbs, bent in elbow, |
| | | | hands slightly flexed |
| | | | Toes separated |
| | | | 4th ventr. still larger than lateral |
| | | | ventr. |
| | | | Scalp vascular plexus visible |
| 21 | 52 | 23 | Hands and feet turned inward |
| | | | Longer fingers |
| | | | Toes distinct but webbed |
| | | | Bending of knees, toes may touch |
| | | | Trunk straight and longer |
| | | | Stubby tail visible |
| 22 | 54 | 26 | Eyelids visible |
| | | | Fingers may overlap |
| | | | Lower limbs rotated, touching feet |
| | | | Very straight trunk |
| | | | 4th ventricle smaller than lateral ventr. |
| | | | Hemispheres recognizable |
| 23 | 56.5 | 29 | Rounded head |
| | | | Limbs increased in length |
| | | | Rotation of lower limbs |
| | | | Forearm ascends to shoulder level |
| | | | Scalp vascular plexus at vertex |

Characteristics of Stages 1-8 were taken from O'Rahilly's study (1987) (Developmental Stages in Human Embryos: Including a Revision of Streeter's “Horizons” and a Survey of the Carnegie Collection. Carnegie Institution of Washington; Washington, DC, USA: 1987) and the characteristics of Stages 9-23 were acquired through a combination of sources, including the HDBR atlas, O'Rahilly (1987), Hill (2007) (Clin. Obstet. Gynecol. 2007; 50: 2-9), and Pietersma (2023) (Hum Reprod, 2023 May 2; 38(5): 820-829). P.O days and embryonic size data utilized within the table were taken from O'Rahilly's study (1987).

In some embodiments, the mammalian embryos generated herein are mouse embryos. Theiler has established numbered stages of murine development. The earliest stages, as applied to (C57BLxCBA)F1 mice, arc described in the “emouse digital atlas” (www.emouseatlas.org) as follows in Table 2.

TABLE 2
Theiler Stages of Development

| Theiler Stage | Dpc* (range) | | Cell number | (C57BL × CBA)Fl mice |
|---|---|---|---|---|
| 1 | 0-0.9 | (0-2.5) | 1 | One-cell egg |
| 2 | 1 | (1-2.5) | 2-4 | Dividing egg |
| 3 | 2 | (1-3.5) | 4-16 (or 8- | Morula |
| | | | 16) | |
| 4 | 3 | (2-4) | 16-40 (or | Blastocyst, inner cell mass apparent |
| | | | 16-32) | |
| 5 | 4 | (3-5.5) | | Blastocyst (zona-free) |
| 6 | 4.5 | (4-5.5) | | Attachment of blastocyst; primary endoderm covers |
| | | | | blastocoelic surface of inner cell mass |
| 7 | 5 | (4.5-6) | | Implantation and formation of egg cylinder; |
| | | | | Ectoplacental cone appears, enlarged epiblast, primary |
| | | | | endoderm lines mural trophectoderm |
| 8 | 6 | (5-6.5) | | Differentiation of egg cylinder. Implantation sites 2 × 3 |
| | | | | mm. Ectoplacental cone region invaded by maternal |
| | | | | blood, Reichert's membrane and proamniotic cavity |
| | | | | form |
| 9a | 6.5 | (6.25-7.25) | | Pre-streak(PS). advanced endometrial reaction, ecto |
| | | | | lacental cone invaded by blood, extraembryonic |
| | | | | ectoderm, embryonic axis visible |
| 9b | | | | Early streak(ES), gastrulation starts, first evidence of |
| | | | | mesoderm |
| 10a | 7 | (6.5-7.75) | | Mid streak (MS), amniotic fold starts to form |
| 10b | | | | Late streak, no bud (LSOB), exocoelom |
| 10c | | | | Late streak, early bud (LSEB), allantoic bud first |
| | | | | appears, node, amnion closing |
| 11a | 7.5 | (7.25-8) | | Neural plate (NP), head process developing, amnion |
| | | | | complete |
| 11b | | | | Late neural plate (LNP), elongated allantoic bud |
| 11c | | | | Early head fold (EHF) |
| 11d | | | | Late head fold (LHF), foregut invagination |
| 12a | 8 | (7.5-8.75) | | 1-4 somites, allantois extends, first branchial arch, |
| | | | | heart starts to form, foregut pocket visible, preotic |
| | | | | sulcus at 2-3 somite stage) |
| 12b | | | | 5-7 somites, allantois contacts chorion at the end of |
| | | | | TS12, Absent 2^{nd} arch, >7 somites |
| 13 | 8.5 | (8-9.25) | | Turning of the embryo, 1^{st} branchial arch has |
| | | | | maxillary and mandibular components, 2^{nd} arch |
| | | | | present; Absent 3rd branchial arch |
| 14 | 9 | (8.5-9.75) | | Formation & closure of ant. neuropore, otic pit |
| | | | | indented but not closed, 3^{rd} branchial arch visible; |
| | | | | Absent forelimb bud |
| 15 | 9.5 | (9-10.5) | | Formation of post. neuropore, |
| | | | | forelimb bud, forebrain vesicle subdivides; |
| | | | | Absent hindlimb bud, Rathke's pouch |
| 16 | 10 | (9.5-10.75) | | Posterior neuropore closes, Formation of hindlimb & |
| | | | | tail buds, lens plate, Rathke's pouch; the indented nasal |
| | | | | processes start |
| | | | | to form; Absent thin & long tail |
| 17 | 10.5 | (10-11.25) | | Deep lens indentation, adv. devel. of brain tube, tail |
| | | | | elongates and thins, umbilical hernia starts to form; |
| | | | | Absent nasal pits |
| 18 | 11 | (10.5-11.25) | | Closure of lens vesicle, nasal pits, cervical somites no |
| | | | | longer visible; |
| | | | | Absent auditory hillocks, anterior footplate |
| 19 | 11.5 | (11-12.25) | | Lens vesicle completely separated from the surface |
| | | | | epithelium, Anterior, but no posterior, footplate. |
| | | | | Auditory hillocks first visible; Absent retinal |
| | | | | pigmentation and sign of fingers |
| 20 | 12 | (11.5-13) | | Earliest sign of fingers, (splayedout), posterior |
| | | | | footplate apparent, retina pigmentation apparent, |
| | | | | tongue well-defined, brain vesicles clear; |
| | | | | Absent 5 rows of whiskers, indented |
| 21 | 13 | (12.5-14) | | Anterior footplate indented, elbow and wrist |
| | | | | identifiable, 5 rows of whiskers, umbilical hernia now |
| | | | | clearly apparent; |
| | | | | Absent hair follicles, fingers separate distally |
| 22 | 14 | (13.5-15) | | Fingers separate distally, only indentations between |
| | | | | digits of the posterior footplate, long bones of limbs |
| | | | | present, hair follicles in pectoral, pelvic and trunk |
| | | | | regions; |
| | | | | Absent open eyelids, hair follicles in cephalic region |

| 23 | 15 | Fingers & Toes separate, hair follicles also in cephalic |
|---|---|---|
| | | region but not at periphery of vibrissae, |
| | | eyelids open; |
| | | Absent nail primordia, fingers 2-5 parallel |
| 24 | 16 | Reposition of umbilical hernia, eyelids closing, fingers |
| | | 2-5 are parallel, nail primordia visible on |
| | | Toes; |
| | | Absent wrinkled skin, fingers & toes joined together |
| 25 | 17 | Skin is wrinkled, eyelids are closed, umbilical hernia is |
| | | gone; |
| | | Absent ear extending over auditory meatus, long |
| | | whiskers |
| 26 | 18 | Long whiskers, eyes barely visible through closed |
| | | eyelids, ear covers auditory meatus |
| 27 | 19 | Newborn Mouse |

*“dpc” indicates days post conception, with the morning after the vaginal plug is found being designated 0.5 dpc or E0.5.

The methods, compositions, and kits herein described can be applied to embryos from any suitable mammalian species, such as: primates, including humans, great apes (e.g., gorillas, chimpanzees, orangutans), old world monkeys, new world monkeys; rodents (e.g., mice, rats, guinea, pigs, hamsters); cats; dogs; lagomorphs (e.g., rabbits); cows; sheep; goats; horses; pigs; and any other livestock, agricultural, laboratory or domestic mammals. The methods and compositions herein described can be applied to an embryo from any non-human mammal, including but not limited to those described above.

### Amnion Cells and Cell Models

Provided herein include amnion cell populations and amnion cell models comprising one or more amnion cell types. The one or more cell types can be selected from the group consisting of amnion epithelial cells, amnion epithelial cells, fibroblasts, macrophages, amnion mesenchymal stem cells, and amnion epithelial stem cells. In some embodiments, the one or more cell types can be characterized by expression or overexpression of specific cell markers identified herein, including amnion epithelial cell markers, amnion mesenchymal cell markers, fibroblast markers, macrophage markers, amnion mesenchymal stem cell markers, and/or amnion epithelial stem cell markers. The amnion cells, cell populations, and cell models can be derived from amnion, such as human amnion, or in vitro generated amnion models. In some embodiments, the amnion cells, cell populations, and cell models can be generated by directed differentiation of pluripotent or multipotent stem cells that exhibit transcriptomic and/or protein expression characteristic of amnion lineages. The amnion cell models and amnion cell populations described herein can comprise amnion cells genetically engineered to express or overexpress one or more of the marker genes identified herein.

In some embodiments, an amnion cell population comprises cells of one or more amnion cell types. As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells. An amnion cell population can comprise one or more cell types selected from amnion epithelial cells, amnion epithelial cells, fibroblasts, macrophages, amnion mesenchymal stem cells, or amnion epithelial stem cells, optionally expanded in vitro, and retaining biomarkers characteristic of their lineage. In some embodiments, an amnion cell population comprises amnion stem cells, including amnion epithelial stem cells, amnion mesenchymal stem cells, or both. In some embodiments, an amnion cell population comprises amnion epithelial cells. In some embodiments, an amnion cell population comprises amnion mesenchymal cells.

In some embodiments, a cell model (e.g., amnion cell model) comprise one or more amnion cell types. For example, an amnion cell model can comprise amnion epithelial cells and/or amnion mesenchymal cells. In some embodiments, an amnion cell model comprises amnion stem cells including amnion epithelial stem cells, amnion mesenchymal stem cells, or both.

In some embodiments, the amnion cells are mammalian amnion cells. In some embodiments, the amnion cells are human amnion cells. The amnion cells can be collected from human amnion samples, such as a first trimester human amnion tissue. For example, the amnion cells can be collected from a human amnion sample corresponding to Carnegie stage (CS) 5, 6, 7, 8, 9, 10, 11, 12, 13, 14, 15, 16, 17, 18, 19, 20, 21, 22, or 23. In some embodiments, the human amnion samples represent 5-9 weeks of pregnancy and corresponding to Carnegie stages (CS) 16, 17, 19, 22 and 23, respectively. Specifically, CS16 embryos have developed limb buds, the otic vesicle, early eye structures and the primitive heart tube, along with forming somites and the neural tube. CS17 embryos have developed hand rays, cartilage, ribs, intercostal muscles, mammary glands and the thymus. By CS19, embryos have developed the cerebral aqueduct, middle cerebral artery, renal artery and tibia. By CS22, the embryonic brain has developed nerve cell clusters and bundles of nerve fibers, and ossification has begun in the clavicle and long bones.

In some embodiments, the amnion cells used herein are collected from in vitro amnion models corresponding to different developmental stages of a natural embryo produced in vivo (e.g., Carnegie stage (CS) 5, 6, 7, 8, 9, 10, 11, 12, 13, 14, 15, 16, 17, 18, 19, 20, 21, 22, or 23). For example, the amnion models can be derived from human pluripotent stem (hPS) cells using two-dimensional (2D) cell cultures, three-dimensional cell cultures, or a microfluidic device known in the art. Pluripotent cells can include any human stem cells. Methods of culturing amnion cells from hPS in different culture media are described in Nature, 2019 September; 573 (7774): 421-425. doi: 10.1038/s41586-019-1535-2; Nat Commun, 2024 Jan. 2; 15 (1): 167. doi: 10.1038/s41467-023-43871-2; Cell Stem Cell, 2022 May 5; 29 (5): 744-759.e6. doi: 10.1016/j.stem.2022.03.014, the contents of which are incorporated herein by reference in their entireties.

The amnion cell model disclosed herein can comprise major cell types and/or subtypes identified in amnion, particularly human amnion across the first trimester of pregnancy. The amnion cell types can be broadly categorized into epithelial, mesenchymal and macrophage lineages. In some embodiments, cell types in human amnion can be categorized into the following cell types or subpopulations of cells: amnion epithelial cells, amnion mesenchymal cells, amnion mesenchymal stem cells, amnion epithelial stem cells, macrophages, and fibroblasts.

In some embodiments, cells obtained from human amnion can be selected for a specific cell type such as amnion epithelial cell, amnion mesenchymal cell, amnion epithelial stem cell or amnion mesenchymal stem cell based on the biomarkers and gene signatures identified herein. In some embodiments, cells along a specific lineage (e.g., epithelial or mesenchymal) can be identified. Different cell types and/or subtypes described herein can be isolated using the biomarkers and gene signatures expressed by the cell type or subtype. Isolated cells or cell populations described herein can be cultured in vitro. Amnion cells or various cell types of amnion cells described herein can be introduced to a subject for cell-based therapies and regenerative medicine application, such as wound healing, treatment of ocular surface disorder and tissue engineering.

Disclosed herein also include genetically engineered cells derived from amnion or one or more cell types of amnion. In some embodiments, the cells are engineered to express or overexpress one or more biomarker genes described herein to modulate differentiation or cell lineage. In some embodiments, the cells can be engineered to express or overexpress one or more biomarker genes (e.g., mesenchymal biomarker genes) to undergo an epithelial to mesenchymal transition. In some embodiments, cells can be engineered to express or overexpress one or more biomarker genes to undergo a mesenchymal to epithelial transition. In some embodiments, engineered cells of specific cell types can be induced by activating or inhibiting a cell type specific pathway. Methods of modulating gene expression level are known in the art and described in more details in the sections below.

### Biomarker Genes and Pathways

Provided herein also include biomarker genes (e.g., cell type specific) and signaling pathways for the identification, diagnosis, prognosis and manipulation of cell properties, for use in a variety of research, diagnostic, and/or therapeutic applications. In some embodiments, up and down regulated genes can be used for therapeutic, diagnostic, or screening methods described herein.

The term “biomarker” commonly denotes a biological molecule, particularly an endogenous biological molecule or a detectable portion thereof, whose qualitative and/or quantitative evaluation in a test object (e.g., a cell, cell population, tissue, organ, or organism) is predictive or informative with respect to one or more aspects of the test object's phenotype and/or genotype. Biomarkers in the context of the present disclosure encompasses nucleic acids, genes, peptides, polypeptides and/or proteins encoded by a given gene, reaction products, metabolites, or detectable portions thereof.

In some embodiments, amnion cells can be categorized into different cell types based on the expression of lineage specific genes or biomarkers. These different cell types can be referred to comprising specific gene or protein signatures.

In certain embodiments, biomarkers include the signature genes or signature gene products as described herein. As used herein a “signature” may encompass any gene or genes, protein or proteins, or epigenetic element(s) whose expression profile or whose occurrence is associated with a specific cell type, subtype, or cell state of a specific cell type or subtype within a population of cells. The signature can be a gene signature, a protein signature, or both. The terms “signature”, “expression profile” can be used interchangeably. Levels of expression or activity or prevalence may be compared between different cell types in order to characterize or identify for the signatures specific for cell (sub) populations. Increased or decreased expression or activity or prevalence of signature genes may be compared between different cells in order to characterize or identify for the specific cell (sub) populations. The detection of a signature in single cells may be used to identify and quantitate for the specific cell (sub) populations. A signature may include a gene or genes, protein or proteins, or epigenetic element(s) whose expression or occurrence is specific to a cell (sub) population, such that expression or occurrence is exclusive to the cell (sub) population. A gene signature as used herein, may thus refer to any set of up- and down-regulated genes that are representative of a cell type or subtype. A gene signature as used herein, may also refer to any set of up- and down-regulated genes between different cells or cell (sub) populations derived from a gene-expression profile. For example, a gene signature may comprise a list of genes differentially expressed in a distinction of interest.

The signature as defined herein can be used to indicate the presence of a cell type, a subtype of the cell type, the state of the microenvironment of a population of cells, a particular cell type population or subpopulation, and/or the overall status of the cell population. Furthermore, the signature may be indicative of cells within a population of cells in vivo. The signatures of the present disclosure can be identified by analysis of expression profiles of single-cells within a population of cells from isolated samples (e.g. human amnion). The presence of cell types or cell states can be determined by cell type specific or cell state specific signatures. The presence of these specific cell types or cell states can be determined by applying the signature genes to bulk sequencing data in a sample.

In some embodiments, levels of expression or activity or prevalence may be compared between the same cells or cell models under different conditions (e.g., with or without a perturbation) in order to evaluate the effect of the perturbation on the gene signatures. Increased or decreased expression or activity or prevalence of signature genes can be determined by comparing the one or more gene signatures or pathways of the cells (or cell models) in the presence of the perturbation with one or more gene signatures or pathways of the cells (or cell models) in the absence of the perturbation.

In some embodiments, a signature can comprise or consist of one or more genes, proteins and/or epigenetic elements, such as for instance 1, 2, 3, 4, 5, 6, 7, 8, 9, 10 or more. In some embodiments, the signature can comprise or consist of two or more genes, proteins and/or epigenetic elements, such as for instance 2, 3, 4, 5, 6, 7, 8, 9, 10 or more. It is to be understood that a signature used herein can include genes or proteins as well as epigenetic elements combined.

In some embodiments, a signature used herein is characterized as being specific for a particular cell type or cell (sub) population if it is upregulated or only present, detected or detectable in that particular cell type or cell (sub) population, or alternatively is downregulated or only absent, or undetectable in that particular cell type or cell (sub) population. Accordingly, in some embodiments, a signature can comprise one or more differentially expressed genes/proteins when comparing different cells or cell (sub) populations, as well as comparing same cell populations treated with different conditions. It is to be understood that “differentially expressed” genes/proteins include genes/proteins which are up- or down-regulated as well as genes/proteins which are turned on or off. When referring to up- or down-regulation, in some embodiments, the up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression can be determined based on common statistical tests, as is known in the art. In some embodiments, differential expression can be determined by comparing expression to the mean or median expression of all expressed genes or to a subset of genes.

Provided herein include pathways and genes differentially expressed in various cell types of amnion (e.g., human amnion). In some embodiments, a gene signature of amnion epithelial cells comprises at least 1, 2, 3, 4 or more of GABRP, KRT18, IGFBP2, IGFBP5, NPY, FN1, ITGB6, VTCN1, CLDN6, STMN1, TYMS, MAD2L1, CDK1, ZWINT, TK1, CDH1, preferably GABRP and KRT18. In some embodiments, a gene signature of amnion epithelial stem cells comprises 1, 2, 3, 4 or more of GABRP, KRT18, IGFBP2, VTCN1, CLDN6, STMN1, TYMS, MAD2L1, CDK1, ZWINT, TK1, CDH1, preferably GABRP and KRT18. In some embodiments, a gene signature of amnion mesenchymal cells comprises 1, 2, 3, 4 or more of MGP, DLK1, VIM, IGFL2, POSTN, COL8A2, COL15A1, COL6A1, COL5A1, ADAMTS9, preferably MGP and VIM. In some embodiments, a gene signature of amnion mesenchymal stem cells comprises 1, 2, 3, 4 or more of STMN1, TYMS, MAD2L1, CDK1, MGP, DLK1, VIM, IGFL2, POSTN, PCLAF, CENPK, CENPM, ZWINT, TK1, preferably MGP, VIM, and PCLAF. In some embodiments, a gene signature of fibroblast cells comprises 1, 2, 3, 4 or more of COL6A1, COL5A1, ADAMTS9, MALATI, DLK1, POSTN, preferably COL6A1 and COL5A1. In some embodiments, a gene signature of macrophage cells comprises 1, 2, 3, or 4 of MRC1, CD36, SRGN, FOLR2, PTPRC, preferably MRC1 and CD36. Expression of individual genes can be shared among different cell types, however, expression of a gene signature may be cell type specific.

Identified herein also include genes and markers related to cell type transitions from one lineage to another. For example, pseudotime analysis methods are used in some embodiments to reveal two primary differentiation trajectories: epithelial and mesenchymal. Based on the UMAP and pseudotime analyses, intermediate cells are identified as serving as bridges between epithelial and mesenchymal lineages, suggesting that an epithelial to mesenchymal transition (EMT) occurs in the amnion. Expression of transcription factors related to EMT in different cell subtypes are also identified. EMT-related transcription factors identified herein include SNAI1, SNAI2, ZEB1, ZEB2, TWIST1, and TWIST2. A subtype of amnion epithelial stem cells are also identified as serving an intermediate state between amnion epithelial and mesenchymal lineages.

Identified herein also include transcripts that change expression in accordance with cell differentiation trajectory. Specifically, along the epithelial-macrophage trajectory, upregulation in the expression levels of epithelial and macrophage marker genes (e.g., GABRP, IGFBP3, MRC1 and CD36) is observed. By contrast, mesenchymal marker genes such as MGP and VIM are downregulated. Along the mesenchymal differentiation trajectory, mesenchymal marker genes such as MGP and VIM are upregulated, whereas epithelial and macrophage marker genes (e.g., GABRP, IGFBP3, MRC1 and CD36) are downregulated. Accordingly, the upregulation and/or downregulation of cell type marker genes can be used to characterize the cell's differentiation trajectory.

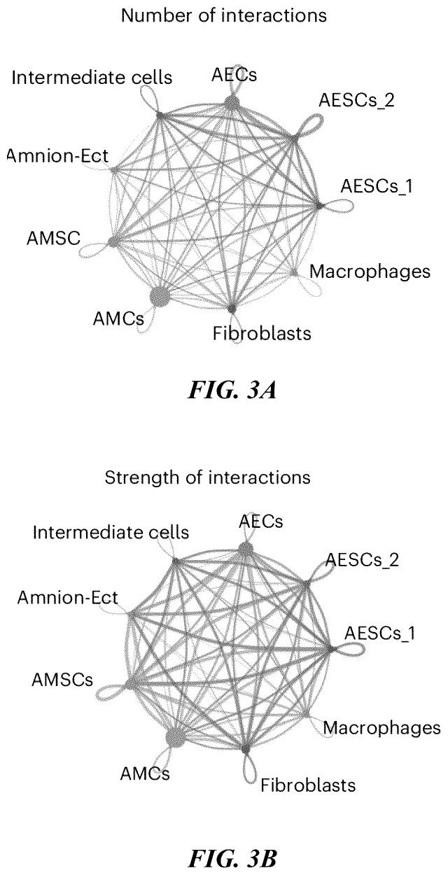

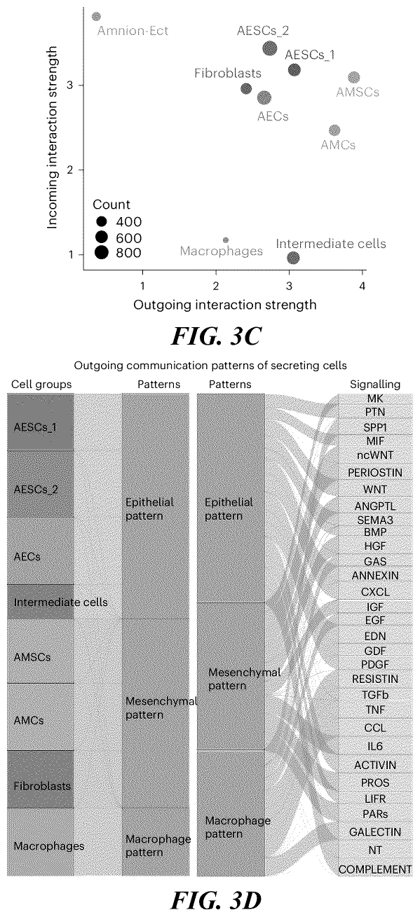

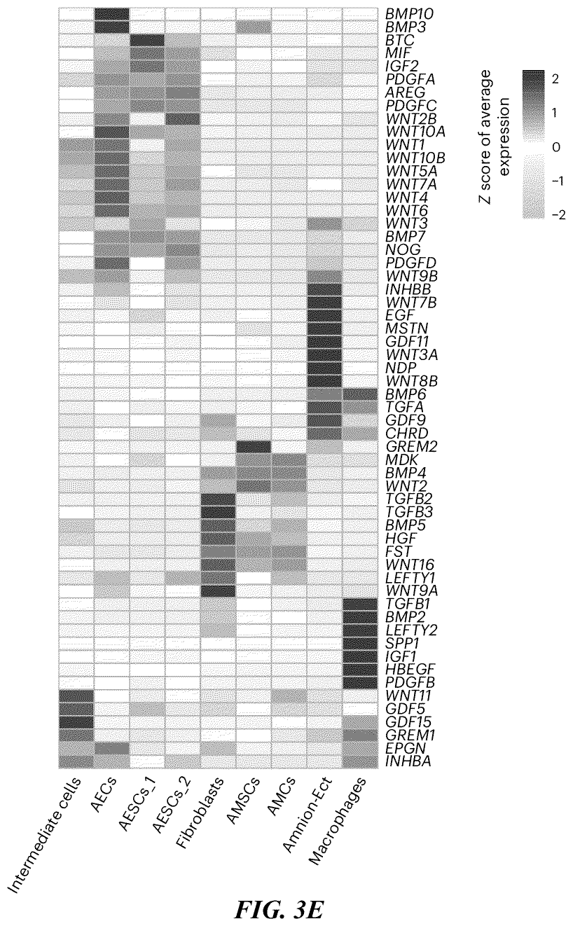

Methods of modulating proliferation, differentiation and/or function of one or more cell types including amnion epithelial cells and amnion mesenchymal cells can include modulating one or more marker genes disclosed herein as well as genes involved in cell type specific signaling pathways. Identified herein include three patterns based on the expression of genes encoding secreted signaling ligands: epithelial pattern, mesenchymal pattern and macrophage pattern. The epithelial pattern includes cells with expression of genes associated with bone morphogenetic protein (BMP), wingless/integrated (WNT), platelet-derived growth factor (PDGF), and growth differentiation factor (GDF) signaling. The mesenchymal pattern includes cells with expression of genes associated with midkine (MDK), non-canonical Wnt (ncWNT), hepatocyte growth factor (HGF), and insulin-like growth factor (IGF). The macrophage pattern include cells with expression of genes associated with secreted phosphoprotein 1 (SPP1) and transforming growth factor beta (TFG-β). Additional signaling pathways and secretion patterns associated with different cells and cell patterns are illustrated and shown in FIG. 3D.

In some embodiments, signaling molecules disclosed herein can be used in cell culture media to promote pluripotent stem cell differentiation. The signaling molecules disclosed can be used to guide embryonic stem cells and induced pluripotent stem cells (iPSCs) to differentiate into specific cell lineages. In some embodiments, treatment of induced pluripotent stem cells with one or more signaling molecules (e.g., BMP4) disclosed herein in a culture medium can result in the upregulation of amnion markers (e.g., amnion markers of early and/or late stages) and promote amnion development. In some embodiments, the one or more signaling molecules include one or more of BMP10, BMP3, BTC, MIF, IGF2, PDGFA, AREG, PDGFC, WNT2B, WNT10A, WNT1, WNT10B, WNT5A, WNT7A, WNT4, WNT6, WNT3, BMP7, NOG, PDGFD, WNT9B, INHBB, WNT7B, EGF, MSTN, GDF11, WNT3A, NDP, WNT8B, BMP6, TGFA, GDF9, CHRD, GREM2, MDK, BMP4, WNT2, TGFB2, TGFB3, BMP5, HGF, FST, WNT16, LEFTY1, WNT9A, TGFB1, BMP2, LEFTY2, SPP1, IGF1, HBEGF, PDGFB, WNT11, GDF5, GDF15, GREM1, EPGN, and INHBA.

Provided herein also include transcription factors identified to be enriched in different stages of amnion development. For example, transcription factors POU5F1, HAND1, ISL1, ID3, MSX1, EPAS1, ID4, PRRX1, EN1, HOXB6, ID1, DLX5, ID2, MYBL2, TCF4, HOXA10, PPARG, MSX2, TBX3, and HES1 are enriched in early amnion development. Transcription factors associated with epithelial cells include TFAP2B, TFAP2A, IRX1, IRX2, and MEIS2. Transcription factors associated with mesenchymal cells include GATA6, HAND2, LMCD1, PITX1, PITX2, SOX6, FOSL2, TWIST1, LEF1, ZEB1, and TBX2.

Amnion mesenchymal cells and amnion epithelial cells at early or late stages can be identified based on their marker gens along with transcription factors. For example, late-stage amnion mesenchymal cells are enriched with mesenchymal markers such as COL15A1 and MGP, along with transcription factors related to EMT including GATA6, TWIST1 and ZEB1. Late-stage amnion epithelial cells are enriched with epithelial markers such as GABRP and IGFBP5, as well as transcription factors TFAP2A and TFAP2B.

Provided herein also include a single-cell qRT-PCR kit comprising primers targeting at least 3, 4, 5, 6, 7, or more (such as all of) the marker genes described in the present disclosure, including for example, GABRP, KRT18, IGFBP2, IGFBP5, NPY, FN1, ITGB6, VTCN1, CLDN6, STMN1, TYMS, MAD2L1, CDK1, ZWINT, TK1, CDH1, MGP, DLK1, VIM, IGFL2, POSTN, COL8A2, COL15A1, COL6A1, COL5A1, ADAMTS9, PCLAF, CENPK, CENPM, COL6A1, MALAT1, MRC1, CD36, SRGN, FOLR2, PTPRC. Preferably, the qRT-PCR kit comprises primers targeting GABRP, IGFBP3, KRT18, MGP, VIM, and CD36. In some embodiments, the single cell qRT-PCR kit comprises primers targeting at any combination of marker genes described herein.

### Marker Detection

In some embodiments, cell types are identified using biomarkers and/or gene signatures described herein. In some embodiments, the downregulation and upregulation of the biomarkers and/or gene signature can be monitored in a population of cells (e.g., amnion cells) in response to a perturbation (e.g., a test agent). In some embodiments, cell groups/types (e.g., amnion epithelial cells, amnion mesenchymal cells, fibroblasts, macrophages, amnion epithelial stem cells, and/or amnion mesenchymal stem cells) expressing different biomarkers can be identified. The amnion cells can be collected from a biological sample (e.g., from a pregnant subject). In some embodiments, the amnion cells are generated from in vitro or ex vivo stem cell-based culturing systems as described herein.

The differentially expressed proteins identified herein can be used as a protein marker to predict the developmental potential of different amnion cells. One or more protein markers as described herein can be detected using any suitable method identifiable to a person skilled in the art. Numerous methods exist in the art for detecting the presence, absence, or amount of a marker gene product (e.g., mRNA and/or protein), as well as its localization in a cell structure or subcellular localization (e.g., nucleus and/or cytoplasm). Marker expression may be assessed by any of a wide variety of well-known methods for detecting expression of a transcribed molecule or a protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification and sequencing methods.

Marker expression can be monitored in a variety of ways, including by detecting mRNA levels, protein levels, or protein activity, any of which can be measured using standard techniques. Detection can involve quantification of the level of gene expression (e.g., genomic DNA, cDNA, RNA, protein, or enzyme activity), or, alternatively, can be a qualitative assessment of the level of gene expression, in particular in comparison with a control level. The type of level being detected will be clear from the context.

In some embodiments, next generation sequencing (e.g., RNA-seq) can be used to analyze total mRNA expression from one (e.g., single-cell RNA-seq) or more cells. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLID sequencing, and nanopore sequencing amongst others. Methods for constructing sequencing libraries are known in the art.

In some embodiments described herein, the method involves single cell RNA sequencing (scRNA-seq). scRNA-seq is a next-generation sequencing method that examines the genomes or transcriptomes of individual cells, providing a high-resolution view of cell-to-cell variation (see, e.g., Haque, A., Engel, J., Teichmann, S. A. et al. A practical guide to single-cell RNA-sequencing for biomedical research and clinical applications. Genome Med 9, 75 (2017). doi.org/10.1186/s13073-017-0467-4).

The single cell sequencing can be high-throughput single cell RNA sequencing. In certain embodiments, the single cell sequencing is a low cost high-throughput single cell RNA sequencing. Not being bound by any particular theory, the single cell RNA sequencing is capable of efficiently and cost effectively sequencing thousands to tens of thousands of single cells. In certain embodiments, single cell RNA sequencing comprises pairing single cells in droplets with oligonucleotides for reverse transcription, wherein the oligonucleotides are configured to provide cell-of-origin specific barcodes uniquely identifying transcripts from each cell and a unique molecular identifier (UMI) uniquely identifying each transcript. In certain embodiments, single cell RNA sequencing comprises pairing single cells in droplets with single microparticle beads coated with oligonucleotides for reverse transcription, wherein the oligonucleotides contain a bead-specific barcode uniquely identifying each bead and a unique molecular identifier (UMI) uniquely identifying each primer. In some aspects of the disclosure, unbiased classifying of cells in a biological sample comprises sequencing the transcriptomes of thousands of cells, preferably tens of thousands of cells (e.g., greater than 1000 cells, or greater than 10,000 cells).

In some embodiments, a transcriptome is sequenced. As used herein, the term “transcriptome” refers to the set of transcripts molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 50%, 55%, 60%, 65%, 70%, 75%, 80%, 85%, 90%, 91%, 92%, 93%, 94%, 95%, 96%, 97%, 98%, 99%, 99.9%, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In some embodiments, assessing the cell (sub) types and states present in an in vitro or ex vivo system may comprise analysis of expression matrices from scRNA-seq data, performing dimensionality reduction, graph-based clustering and deriving list of cluster-specific genes in order to identify cell types and/or states present in the system. The clustering and gene expression matrix analysis allow for the identification of key genes in the ex vivo system, such as differences in the expression of key transcription factors.

In some embodiments, dimension reduction is used to cluster single cells based on differentially expressed genes. In certain embodiments, the dimension reduction technique may be, but is not limited to, Uniform Manifold Approximation and Projection (UMAP) t-SNE, or PHATE (see, e.g., Becht et al., Evaluation of UMAP as an alternative to t-SNE for single-cell data, bioRxiv 298430; doi.org/10.1101/298430; Becht et al., 2019, Dimensionality reduction for visualizing single-cell data using UMAP, Nature Biotechnology volume 37, pages 38-44; and Moon et al., PHATE: A Dimensionality Reduction Method for Visualizing Trajectory Structures in High-Dimensional Biological Data, bioRxiv 120378; doi: doi.org/10.1101/120378).

In some embodiments, activity of a particular gene is characterized by a measure of gene transcript (e g., mRNA), by a measure of the quantity of translated protein, or by a measure of gene product activity.

In some embodiments, detecting or determining expression levels of a marker and functionally similar homologs thereof, including a fragment or genetic alteration thereof (e.g., in regulatory or promoter regions thereof) comprises detecting or determining RNA levels for the marker of interest. In some embodiments, one or more cells from the synthetic embryo structure can be obtained and RNA is isolated from the cells. In some embodiments, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated. It is also be possible to obtain cells from, e.g., the synthetic embryo cells and culture the cells in vitro, such as to obtain a larger population of cells from which RNA can be extracted. Methods for establishing cultures of non-transformed cells, i.e., primary cell cultures, are known in the art. In some embodiments, cells can be dissociated (e.g., by enzymatic or mechanical means), and isolated by methods known in the art (e g., Fluorescence-Activated Cell Sorting, Microfluidics, etc.)

When isolating RNA from, e.g., synthetic embryos at various developmental stages and/or cells comprising said synthetic embryos, it may be important to prevent any further changes in gene expression after the tissue or cells has been removed from the subject. Changes in expression levels are known to change rapidly following perturbations, e g., heat shock or activation with lipopolysaccharide (LPS) or other reagents. In addition, the RNA in the tissue and cells may quickly become degraded. Accordingly, in some embodiments, the tissue or cells obtained from a subject is snap frozen as soon as possible.

RNA can be extracted from cells by a variety of methods, e.g., the guanidium thiocyanate lysis followed by CsCl centrifugation. Methods for obtaining RNA from single-cells are also known in the art. The RNA sample can then be enriched in particular species. In some embodiments, poly (A)+RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.). In some embodiments, the RNA population is enriched in marker sequences. Enrichment can be undertaken, e.g., by primer-specific cDNA synthesis, or multiple rounds of linear amplification based on cDNA synthesis and template-directed in vitro transcription.

The population of RNA, enriched or not in particular species or sequences, can further be amplified. As defined herein, an “amplification process” increases the number of copies of a polynucleotide (e.g., RNA). For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the disclosed methods to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4:80-84 (1994). Real time PCR may also be used. Other known amplification methods which can be utilized herein include but are not limited to the so-called “NASBA” or “3 SR” technique described in PNAS USA 87:1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42:9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e g., Kwoh et al., Proc. Natl. Acad. Sci. USA 86, 1173 (1989)). Many techniques are known in the state of the art for determining absolute and relative levels of gene expression, commonly used techniques suitable for use in the disclosed methods include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Nonradioactive labels such as digoxigenin may also be used. In some embodiments, the probe is labeled with a fluorescence moiety.

Alternatively, mRNA expression can be detected on a DNA array, chip or a microarray. Labeled nucleic acids of a test sample obtained from a subject may be hybridized to a solid surface comprising marker DNA. Positive hybridization signal is obtained with the sample containing marker transcripts. Methods of preparing DNA arrays and their use are well known in the art (see, e.g., U.S. Pat. Nos. 66,186,796; 6,379,897; 6,664,377; 6,451,536; 548,257; U.S. 20030157485). Serial Analysis of Gene Expression (SAGE) can also be performed (See for example U.S. patent application No. 20030215858).

In some embodiments, the activity or level of a lineage marker protein can be detected and/or quantified by detecting or quantifying the expressed marker polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like.

Described below are non-limiting examples of techniques that may be used to detect marker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-marker protein antibodies (unlabeled) are then brought into contact with the support and assayed by a secondary immunological reagent, such as labeled protein A or anti-immunoglobulin (suitable labels including ¹²⁵I, horseradish peroxidase, alkaline phosphatase, fluorophore). Chromatographic detection may also be used.

Immunohistochemistry may be used to detect expression of marker protein. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-marker protein antibodies, such as intrabodies, may also be used for imaging purposes, for example, to detect the presence of marker protein in cells or, e.g., an EP structure. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

Antibodies that may be used to detect marker protein include any antibody, whether natural or synthetic, full length or a fragment thereof, monoclonal or polyclonal, that binds sufficiently strongly and specifically to the marker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the marker protein relative to other proteins, such as related proteins.

Antibodies are commercially available or can be prepared by methods known in the art. Antibodies and derivatives thereof that may be used encompass polyclonal or monoclonal antibodies, chimeric, human, humanized, primatized (CDR-grafted), veneered or single-chain antibodies as well as functional fragments, i.e., marker protein binding fragments, of antibodies. For example, antibody fragments capable of binding to a marker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain. In some embodiments, agents that specifically bind to a marker protein other than antibodies are used, such as peptides. Peptides that specifically bind to a marker protein can be identified by any means known in the art. For example, specific peptide binders of a marker protein can be screened for using peptide phage display libraries.

In some embodiments, amnion cells can be engineered to over-express one or more genes of interest such as one or more genes differentially abundant in amnion epithelial stem cells or amnion mesenchymal stem cells. In some embodiments, the method can comprise providing an expression construct comprising a nucleic acid encoding a gene of interest, and introducing the expression construct into one or more target amnion cell in a manner permitting expression of the introduced construct in the one or more target amnion cells, thereby generating at least one engineered amnion cell. The target amnion can be any cell type identified herein including amnion epithelial cell, amnion mesenchymal cell, fibroblast, macrophage, amnion epithelial stem cell, or amnion mesenchymal stem cell. The expression control element can comprise a promoter, an enhancer, a 5′ un-translated region, a 3′ un-translated region, or any combination thereof. The promoter can be a ubiquitous promoter. The promoter can be a constitutive or an inducible promoter.

“Genetic construct” or “construct” as used herein shall be given their ordinary meanings and can also refer to nucleic acids that comprise a nucleotide sequence which encodes a gene product (e.g., an RNA and/or a protein). The nucleic acid can comprise at least one regulatory element for expression (e.g., an expression control element). The nucleic acid can comprise a vector, such as a viral vector. In some embodiments, the vector can comprise an adenovirus vector, an adeno-associated virus vector, an Epstein-Barr virus vector, a Herpes virus vector, an attenuated HIV vector, a retroviral vector, a vaccinia virus vector, or any combination thereof. In some embodiments, the vector can comprise an RNA viral vector. In some embodiments, the vector can be derived from one or more negative-strand RNA viruses of the order Mononegavirales. In some embodiments, the vector can be a rabies viral vector. Many such vectors useful for transferring exogenous genes into mammalian cells are available. The vectors may be episomal, e.g. plasmids, virus-derived vectors such cytomegalovirus, adenovirus, etc., or may be integrated into the cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Retroviral vectors can be “defective”, i.e. unable to produce viral proteins required for productive infection. Replication of the vector can require growth in the packaging cell line. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

In addition to viral vectors, a variety of additional tools have been developed that can be used for the incorporation of exogenous genes into cells. One such method that can be used for incorporating polynucleotides encoding target genes into cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems include the piggybac transposon (described in detail in, e.g., WO 2010/085699) and the sleeping beauty transposon (described in detail in, e.g., US2005/0112764), the disclosures of each of which are incorporated herein by reference.

As used herein, the term “expression vector” or “construct” refers to a vector that directs expression of an RNA or polypeptide (e.g., E-cadherin) from nucleic acid sequences contained therein linked to transcriptional regulatory sequences on the vector. The sequences can be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Gene products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

In some embodiments, the vectors can include a regulatory sequence that allows, for example, the translation of multiple proteins from a single mRNA. Non-limiting examples of such regulatory sequences include internal ribosome entry site (IRES) and 2A self-processing sequence. In some embodiments, the 2A sequence is a 2A peptide site from foot-and-mouth disease virus (F2A sequence). In some embodiments, the F2A sequence has a standard furin cleavage site. In some embodiments, the vector can also comprise regulatory control elements known to one of skill in the art to influence the expression of the RNA and/or protein products encoded by the polynucleotide within desired cells of the subject. In some embodiments, functionally, expression of the polynucleotide is at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is a ration and the splice donor and splice acceptor sequences that regulate the splicing of said intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited, to the cytomegalovirus (CMV) immediate early promoter/enhancer sequences, the Rous sarcoma virus (RSV) promoter/enhancer sequences and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide m response to a signal or stimuli is also referred to as an “inducible element” (that is, it is induced by a signal). Particular examples include, but are not limited to, a hormone (for example, steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

A promoter may promote ubiquitous expression or tissue-specific expression of an operably linked nucleic acid (e.g., engineered nucleic acid) sequence from any species, including humans. In some embodiments, the promoter is a eukaryotic promoter. Non-limiting examples of eukaryotic promoters include TDH3, PGK1, PKC1, TDH2, PYK1, TPI1, AT1, CMV, EF1 alpha, SV40, PGK1 (human or mouse), Ubc, human beta actin, CAG, TRE, UAS, Ac5, Polyhedrin, CaMKIIa, GAL1, GAL10, TEF1, GDS, ADH1, CaMV35S, Ubi, H1, and U6, as would be known to one of ordinary skill in the art (see, e.g., Addgene website: blog.addgene.org/plasmids-101-the-promoter-region).

Non-limiting examples of ubiquitous promoters include tetracycline-responsive promoters (under the relevant conditions), CMV (EF1 alpha, a SV40 promoter, PGK1, Ubc, CAG, human beta actin gene promoter, a RSV promoter, an EFS promoter, and a promoter comprising an upstream activating sequence (UAS). In certain embodiments, the promoter is a mammalian promoter.

In some embodiments, a promoter of the present disclosure is suitable for use in AAV vectors. See, e.g., U.S. Patent Application Publication No. 2018/0155789, which is hereby incorporated by reference in its entirety for this purpose.

Non-limiting examples of constitutive promoters include CP1, CMV, EF1 alpha, SV40, PGK1, Ubc, human beta actin, beta tubulin, CAG, Ac5, Rosa26 promoter, COL1A1 promoter, polyhedrin, TEF1, GDS, CaM3 5S, Ubi, H1, U6, red opsin promoter (red promoter), rhodopsin promoter (rho promoter), cone arrestin promoter (car promoter), rhodopsin kinase promoter (rk promoter). In some instances, the constitutive promoter is a Rosa26 promoter. In some instances, the constitutive promoter is a COL1A1 promoter.

An “inducible promoter” is one that is characterized by initiating or enhancing transcriptional activity when in the presence of, influenced by, or contacted by an inducing agent. An inducing agent may be endogenous or a normally exogenous condition, compound, agent, or protein that contacts an engineered nucleic acid (e.g., engineered nucleic acid) in such a way as to be active in inducing transcriptional activity from the inducible promoter. In certain embodiments, an inducing agent is a tetracycline-sensitive protein (e.g., tTA or rtTA, TetR family regulators). Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (TetR, e.g., SEQ ID NO: 26, or TetRKRAB, e.g., SEQ ID NO: 27), a tetracycline operator sequence (tetO) and a tetracycline transactivator fusion protein (tTA), and a tetracycline operator sequence (tetO) and a reverse tetracycline transactivator fusion protein (rtTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), pH-regulated promoters, and light-regulated promoters. A non-limiting example of an inducible system that uses a light-regulated promoter is provided in Wang et al., Nat. Methods. 2012 Feb. 12; 9 (3): 266-9. Additional non-limiting examples of inducible promoters include mifepristone-responsive promoters (e.g., GAL4-E1b promoter) and coumermycin-responsive promoters. See, e.g., Zhao et al., Hum Gene Ther. 2003 Nov. 20; 14 (17): 1619-29.

A “reverse tetracycline transactivator” (“rtTA”), as used herein, is an inducing agent that binds to a TRE promoter (e.g., a TRE3G, a TRE2 promoter, or a P tight promoter) in the presence of tetracycline (e.g., doxycycline) and is capable of driving expression of a transgene that is operably linked to the TRE promoter. rtTAs generally comprise a mutant tetracycline repressor DNA binding protein (TetR) and a transactivation domain (see, e.g., Gossen et al., Science. 1995 Jun. 23; 268 (5218): 1766-9 and any of the transactivation domains listed herein). The mutant TetR domain is capable of binding to a TRE promoter when bound to tetracycline. See, e.g., U.S. Provisional Application No. 62/738,894, entitled MUTANT REVERSE TETRACYCLINE TRANSACTIVATORS FOR EXPRESSION OF GENES, which was filed on Sep. 28, 2018, under attorney docket number H0824.70300US00, and is herein incorporated by reference in its entirety.

In some embodiments, amnion cells can be engineered to knock down one or more target genes such as one or more genes differentially expressed in different amnion cell types. Various gene knockdown methods can be employed to terminate or reduce the expression of a specific gene, including, for example, RNA interference (RNAi), antisense oligonucleotides, and CRISPR technology. In some embodiments, the method comprises introducing into a target amnion cell a nucleic acid targeting a gene of interest. The nucleic acid molecule can be single-stranded or double-stranded (e.g., a sense strand and an antisense strand), and comprise a sequence portion corresponding (identical or complementary) to the target gene.

Physical methods for introducing a polynucleotide into a cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). In some embodiments, the nucleic acids described herein are injected into an embryo.

Chemical means for introducing a polynucleotide into a cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a cell. In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution.

Nucleic acids described herein can be introduced into cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12 (8): 861-70 (2001).

In some aspects, non-viral methods can be used to deliver a nucleic acid described herein into a cell. In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition. Exemplary methods of nucleic acid delivery using a transposon include a Sleeping Beauty transposon system (SBTS) and a piggyBac (PB) transposon system. In some embodiments, an engineered described herein are generated by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

### Applications

The methods, amnion cells, and biomarkers of the present disclosure can be applied in multiple ways. Some exemplary applications are described further below.

In some embodiments, the methods, amnion cells, and biomarkers disclosed herein can have a variety of applications including, e.g., investigating mechanisms of embryonic and/or amniotic development, screening testing agents, generating and utilizing amnion-derived cell populations and biomaterials for research and regenerative medicine and therapeutic applications.

In some embodiments, the methods and biomarkers/gene signatures described herein can be used for identifying various amnion cell types, including amnion epithelia cells, amnion epithelia stem cells, amnion mesenchymal cells, amnion mesenchymal stem cells, fibroblast and macrophage cells. In some embodiments, a method of identifying amnion epithelia cells comprises detecting in a cell population one or more genes or gene expression products selected from the group consisting of: GABRP, KRT18, IGFBP2, IGFBP5, NPY, FN1, ITGB6, VTCN1, CLDN6, STMN1, TYMS, MAD2L1, CDK1, ZWINT, TK1, CDH1, and a combination thereof, optionally, GABRP and KRT18. In some embodiments, a method of identifying amnion epithelia stem cells comprises detecting in a cell population one or more genes or gene expression products selected from the group consisting of: GABRP, KRT18, IGFBP2, VTCN1, CLDN6, STMN1, TYMS, MAD2L1, CDK1, ZWINT, TK1, CDH1, and a combination thereof, preferably GABRP and KRT18. In some embodiments, a method of identifying amnion mesenchymal cells comprises detecting in a cell population one or more genes or gene expression products selected from MGP, DLK1, VIM, IGFL2, POSTN, COL8A2, COL15A1, COL6A1, COL5A1, ADAMTS9, or a combination thereof, optionally MGP and VIM. In some embodiments, a method of identifying amnion mesenchymal stem cells comprises detecting in a cell population one or more genes or gene expression products selected from STMN1, TYMS, MAD2L1, CDK1, MGP, DLK1, VIM, IGFL2, POSTN, PCLAF, CENPK, CENPM, ZWINT, TK1, or a combination thereof, optionally MGP, VIM, and PCLAF. In some embodiments, a method of identifying amnion fibroblast cells comprises detecting in a cell population one or more genes or gene expression products selected from COL6A1, COL5A1, ADAMTS9, MALAT1, DLK1, POSTN, preferably COL6A1 and COL5A1. In some embodiments, a method of identifying amnion macrophage cells comprises detecting in a cell population one or more genes or gene expression products selected from MRC1, CD36, SRGN, FOLR2, PTPRC, preferably MRC1 and CD36. The cell population can be obtained from a human amnion sample or from in vitro generated amnion models. The methods herein described can further comprise obtaining a single cell gene expression profile for each of the one or more cells comprising gene expression measurements for a set of genes comprising at least 2, 3, 4, 5, or more of the marker genes described herein. In some embodiments, the single cell gene expression profile can be obtained by performing single cell sequencing such as single cell RNA sequencing. In some embodiments, obtaining a single cell gene expression profile for each of the one or more cells comprises performing single cell RT-qPCR targeting a set of marker genes described herein. In some embodiments, the method comprises obtaining a transcriptome of the one or more cells.

In some embodiments, identifying amnion cell type based on the gene expression profile can comprise classifying each of the one or more cells between two or more cell type classes. For example, the two or more cell type classes can comprise at least a first class, a second class, a third class, a fourth class, a fifth class or a sixth class, wherein cells classified in the classes can be identified to be amnion epithelial cell, amnion mesenchymal cell, fibroblast, macrophage, amnion mesenchymal stem cell, and/or amnion epithelial stem cell. In some embodiments, the method can comprise applying a computational classifier, such as a machine learning model, to the gene expression profile to classify each of the one or more cells between the two or more cell type classes. The computational classifier may optionally have been subjected to multiple rounds of training in order to tune model parameters and/or optimize performance of the computational classifier on the training samples. In some embodiments, the computational classifier comprises a machine learning model. For example, the classifier may comprise a support vector machine (SVM), a decision tree (or ensemble of decision trees) or a logistic regression classifier. In some embodiments, the classifier comprises an ensemble of decision trees trained using gradient boosting.

In some embodiments, the methods and cells/cell models disclosed herein can be used to identify impact or effect of perturbations or stimuli or mutations on embryonic and/or amniotic development. Disclosed herein include methods for detecting perturbation-induced changes in amnion cells. In some embodiments, the method can comprise introducing a perturbation to one or more amnion cells or cell models described herein, determining a perturbation-induced change in one or more gene signatures or pathways of the cells or cell models, and evaluating the effect of the perturbation on the cells or cell models by detecting expression of one or more amnion epithelial cell markers, amnion mesenchymal cell markers, fibroblast markers, macrophage markers, amnion mesenchymal stem cell markers, and/or amnion epithelial stem cell markers described herein. The method can comprise detecting the up/down-regulation of the one or more cell marker genes and/or genes associated with various epithelial or mesenchymal signaling pathways identified herein, as well as the up/down-regulation of one or more transcription factors identified herein. In some embodiments, the detection comprises comparing the one or more gene signatures or pathways of the cells/cell models in the presence of the perturbation with one or more gene signatures or pathways of the cells/cell models in the absence of the perturbation. In some embodiments, determining the perturbation-induced change in the one or more gene signatures or pathways of the cells/cell models comprises performing single cell sequencing.

The perturbation can be a chemical perturbation or a physical perturbation. The perturbations may comprise one or more genetic perturbation. For example, the perturbation can comprise gene knock-down, gene knock-out, gene activation, gene insertion, and/or regulatory element deletion. In some embodiments, the perturbation can comprise genome-wide perturbation. The perturbation may comprise one or more epigenetic or epigenomic perturbation. The perturbation can be a single-order perturbation. Alternatively, the perturbation can comprise combinatorial perturbations. The perturbations can be of a selected group of targets based on similar pathways or network of targets. In some embodiments, the perturbation can target one or more proteins/genes identified to be differentially expressed in different amnion cell types/subtypes. In some embodiments, a transcription factor (e.g., SNAI1, SNAI2, ZEB1, ZEB2, TWIST1, and TWIST2) is targeted. In some embodiments, an epithelial or macrophage marker gene is targeted, including for example GABRP, IGFBP3, MRC1 and CD36. In some embodiments, a mesenchymal marker (e.g., MGP and VIM) is targeted. In some embodiments, the perturbation can target genes associated with one or more signaling pathways identified herein.

In some embodiments, the perturbation can be introduced via a test agent such as a chemical agent or biological agent. Examples of test agents include, but are not limited to, an antibiotic, a small molecule, a growth factor, a hormone or a derivative thereof, a steroid or a derivative thereof, infectious agents (e.g., infections, viruses, parasites and other bacterial illnesses), and toxic chemicals such as organic mercury, lead, pesticides, and herbicides. In some embodiments, the test agent is a teratogen. A teratogen is a substance that interferes with normal fetal development and causes congenital disabilities. Examples of teratogens include, but are not limited to, drugs (e.g., antiepileptic drugs, antimicrobials, anticoagulants, antithyroid medications, vitamin A, or hormonal medication), alcohol, recreational drugs, chemicals and toxic substances. In some embodiments, the perturbation is an exposure of amnion cells or cell models to a drug candidate. Drug candidates can comprise small molecules, hormones, growth factors, peptides, proteins, nucleic acids, or any combination thereof. Therefore, in some embodiments, the methods disclosed herein can be used for screening perturbations, including compounds or agents or conditions. The method can comprise performing the method as described herein, wherein the perturbation comprises contacting the amnion cells or cell models to each compound or agent or condition, and determining a perturbation-induced change in one or more gene signatures or pathways described herein.

In some embodiments, the perturbation can be an agent capable of inducing cell differentiation. For example, the agent can be a cytokine, a transcription factor, and/or a signaling molecule. In some embodiments, the agent can be any of the transcription factors and signaling molecules identified herein. Introducing the perturbation to the amnion cell model or amnion cells can comprise culturing the amnion cells in the presence of the agent. The effect of the agent on the amnion cells can be evaluated by detecting an expression level of the one or more amnion markers in the presence of the agent and comparing it to the expression level of the same amnion markers in the absence of the agent.

In some embodiments, the perturbation can be an increase or decrease of temperature across a gradient, addition or subtraction of energy, electromagnetic energy, or ultrasound. Other perturbations include exposure to light, movement, agitation, exposure to cellular material derived from other tissues, organisms, or microorganisms, or diseased cell lines.

In some embodiments, the perturbation can be introduced with RNA interference or a CRSPR-Cas system to a target cell. In some embodiments, the perturbation can comprise performing single or combinatorial CRISPR-Cas-based perturbation with a genome-wide library of guide RNAs. For example, each guide RNA can be associated with a unique perturbation barcode. Each gRNA may be co-delivered with a reporter mRNA comprising the unique perturbation barcode. The method may comprise an array-format or pool-format perturbation.

In some embodiments, introducing a perturbation to a target cell comprises contacting the perturbation (e.g., a test agent or a drug candidate) with a target cell in a culture media or culturing a target cell in a culture media in the presence of the perturbation or under a perturbed condition such as under an elevated temperature, pressure, pH value or other modified physiological conditions. The term “physiological conditions,” as used herein, can refer to a range of chemical (e.g., pH, ionic strength), biochemical (e.g., enzyme concentrations), and physical (e.g., temperature, pressure) conditions that can be encountered in intracellular and extracellular fluids of tissues, such as, for example, in the intracellular and extracellular fluids of a subject. For most cells and tissues, the physiological pH ranges from about 7.0 to about 7.5, the physiological ionic strength ranges from about 50 mM to about 400 mM, the physiological temperature ranges from about 20° C. to about 42° C., and the physiological pressure ranges from about 925 mbar to about 1050 mbar.

In some embodiments, a perturbation can induce an adverse effect on the amniotic development by increasing or decreasing one or more differentially expressed marker genes or marker genes associated with one or more signaling pathways identified herein. For example, the perturbation can induce an adverse effect on the amniotic development by decreasing or increasing the expression level of one or more differentially expressed marker genes by about, at least, at least about 10%, 15%, 20%, 25%, 30%, 35%, 40%, 45%, 50%, 55%, 60%, 65%, 70%, 75%, 80%, 85%, 90%, 95%, or greater, compared to that in a control amnion cell in the absence of the perturbation. In some embodiments, the perturbation is considered harmful or toxic to the embryonic development if the perturbation decreases the expression level of one or more marker genes by about, at least, at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values). In some embodiments, the perturbation is considered harmful or toxic to the embryonic development if the perturbation increases the expression level of one or more marker genes by about, at least, at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values).

In some embodiments, a perturbation can promote or induce amniotic development by increasing or decreasing one or more differentially expressed marker genes or marker genes associated with one or more signaling pathways identified herein. For example, the perturbation can promote or induce amniotic development by decreasing or increasing the expression level of one or more differentially expressed marker genes by about, at least, at least about 10%, 15%, 20%, 25%, 30%, 35%, 40%, 45%, 50%, 55%, 60%, 65%, 70%, 75%, 80%, 85%, 90%, 95%, or greater, compared to that in a control amnion cell in the absence of the perturbation. In some embodiments, a perturbation can promote or induce amniotic development by upregulating of early and/or late amnion marker expression, including for example COL15A1, MGP, GABRP and IGFBP5. In some embodiments, a perturbation can promote or induce amniotic development by upregulating transcription factors including for example GATA6, TWIST1, ZEBI, TFAP2A and TFAP2B. In some embodiments, the perturbation is considered beneficial to the embryonic development if the perturbation increases the expression level of one or more marker genes (e.g., amnion markers of early and/or late stages) by about, at least, at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values).

Provided herein also include methods of differentiating stem cells into amnion cells. In some embodiments, the method can comprise culturing pluripotent stem cells (e.g., human induced pluripotent stem (iPS) cells) in the presence of one or more agents capable of modulating (e.g., increasing or decreasing) expression of one or more amnion marker genes identified herein in a culture medium under a condition allowing the pluripotent stem cells to differentiate into amnion cells. The culture medium comprises a basal culture medium providing essential salts, amino acids, vitamins, and carbohydrates. In some embodiments, the basal culture medium comprises Dulbecco's Modified Eagle Media (DMEM), DMEM Nutrient Mixture 12 (DMEM/F12), a non-human serum or serum substitute thereof, a reducing agent, an antibiotic or an antimicrobial, L-glutamine or an analogue thereof, or any combination thereof. In some embodiments, the non-human serum or serum substitute comprises fetal bovine serum, bovine serum albumin, KnockOut™ Serum Replacement, or any combination thereof. The reducing agent can comprise, for example, beta-mercaptoethanol (BME), N-acetyl-L-cysteine, dithiothreitol (DTT), or any combination thereof. In some embodiments, the antibiotic comprises Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. The basal culture medium can be supplemented with sodium pyruvate, N2, B27 or other agents to induce specific signaling pathways. In some embodiments, the culture medium comprises a basal pluripotent stem cell medium (e.g., 4CL medium), N2 supplement, B27 supplement, sodium pyruvate, non-essential amino acids, antibiotic, L-ascorbic acid, epigenetic modifiers such as 3-deazaneplanocin A (DZNep) and trichostatin A (TSA), small molecules that target signaling pathways such as PD032590 and IWR-1, growth factors/cytokines such as leukemia inhibitory factor (LIF) and activin A.

In some embodiments, the culture medium comprises the one or more agents capable of modulating (e.g., increasing or decreasing) expression or activity of one or more amnion marker genes identified herein. The one or more amnion marker genes can include one or more of GABRP, KRT18, IGFBP2, IGFBP5, NPY, FN1, ITGB6, VTCN1, CLDN6, STMN1, TYMS, MAD2L1, CDK1, ZWINT, TK1, CDH1, MGP, DLK1, VIM, IGFL2, POSTN, COL8A2, COL15A1, COL6A1, COL5A1, ADAMTS9, PCLAF, CENPK, CENPM, COL6A1, MALAT1, MRC1, CD36, SRGN, FOLR2, or PTPRC. The one or more agents capable of modulating expression or activity of the one or more amnion marker genes can comprise one or more cytokines, growth factors, hormones, transcription factors, signaling molecules, metabolites or small molecules disclosed herein. The transcription factors can include POU5F1, HAND1, ISL1, ID3, MSX1, EPAS1, ID4, PRRX1, EN1, HOXB6, ID1, DLX5, ID2, MYBL2, TCF4, HOXA10, PPARG, MSX2, TBX3, and/or HES1. In some embodiments, the transcription factors include TFAP2B, TFAP2A, IRX1, IRX2, and/or MEIS2. In some embodiments, the transcription factors include GATA6, HAND2, LMCD1, PITX1, PITX2, SOX6, FOSL2, TWIST1, LEF1, ZEBI, and/or TBX2. In some embodiments, the signaling molecules can include one or more of BMP10, BMP3, BTC, MIF, IGF2, PDGFA, AREG, PDGFC, WNT2B, WNT10A, WNT1, WNT10B, WNT5A, WNT7A, WNT4, WNT6, WNT3, BMP7, NOG, PDGFD, WNT9B, INHBB, WNT7B, EGF, MSTN, GDF11, WNT3A, NDP, WNT8B, BMP6, TGFA, GDF9, CHRD, GREM2, MDK, BMP4, WNT2, TGFB2, TGFB3, BMP5, HGF, FST, WNT16, LEFTY1, WNT9A, TGFB1, BMP2, LEFTY2, SPP1, IGF1, HBEGF, PDGFB, WNT11, GDF5, GDF15, GREM1, EPGN, and INHBA. The one or more agents or any analogue thereof can be provided in a concentration from about 10 ng/ml to about 50 ng/ml, including for example 10 ng/ml, 20 ng/ml, 30 ng/ml, 40 ng/ml, 50 ng/ml, or a number or a range between any two of these values. In some embodiments, the one or more agents comprises BMP4. Accordingly, in some embodiments, the method of differentiating stem cells into amnion cells comprises contacting the human induced pluripotent stem (iPS) cells with BMP4.

The method can further comprise identifying suitable agents capable of promoting or modulating cell differentiation by detecting the expression level of the one or more amnion gene markers, transcription factors, and/or signaling molecules identified herein. The method can comprise comparing the expression level of the one or more amnion gene markers of the cultured cells in the presence of an agent to the expression level of the same one or more amnion gene markers in the absence of the agent.

Provided herein also include differentiated amnion cells obtainable by any of the methods disclosed herein. The differentiated amnion cells can be amnion epithelial cells, amnion epithelial cells, fibroblasts, macrophages, amnion mesenchymal stem cells, and/or amnion epithelial stem cells. In some embodiments, the differentiated amnion cells comprise amnion stem cells, including amnion epithelial stem cells, amnion mesenchymal stem cells, or both.

Provided herein also include an isolated human amnion epithelial cell population, an isolated human amnion mesenchymal cell population, an isolated human amnion stem cell populations including an isolated human amnion epithelial stem cell population, an isolated human amnion mesenchymal stem cell population, or both. The isolated human amnion epithelial cell population can be enriched for late-stage epithelial identify, and comprise cells that express epithelial markers such as GABRP and IGFBP5 and transcription factors such as TFAP2A and TFAP2B. The isolated human amnion mesenchymal cell population can be enriched for late-stage mesenchymal identity, and comprise cells that express mesenchymal markers such as COL15A1, MGP and VIM, and EMT-related transcription factors such as GATA6, TWIST1 and ZEB1. In some embodiments, the human amnion mesenchymal cell population are negative or low for epithelial markers. In some embodiments, the cells are obtained from first trimester amnion or from directed differentiation.

The isolated amnion cells disclosed herein can have a variety of applications including, e.g., investigating mechanisms of embryonic and particularly amniotic development and in a variety of research, diagnostic, and/or therapeutic applications. For example, because of their immunogenicity, anti-inflammatory and antimicrobial properties, amnion cells derived herein, particularly amnion stem cells, can be used for various therapeutic application, such as wound healing, tissue engineering/bioactive products, eye diseases, and immunomodulatory therapies.

Provided herein also includes a cell-based therapeutic using amnion-derived cells described herein, including for example amnion epithelial cells, amnion mesenchymal cells, amnion epithelial stem cells, and/or amnion mesenchymal stem cells. In some embodiments, a cell-based therapeutic includes engraftment of amnion cells of the present disclosure. As used herein, the term “engraft” or “engraftment” refers to the process of cell incorporation into a tissue of interest in vivo through contact with existing cells of the tissue. In some embodiments, the amnion-derived cells can be provided in an amnion cell sheet. As used herein, “an amnion cell sheet” refers to a contiguous, substrate-free or substrate-supported layer of cells comprising amnion-derived cells and/or amnion-lineage cells differentiated in vitro. An amnion cell sheet typically includes an extracellular matrix (ECM) component deposited by the cells and/or an underlying acellular membrane, and is adapted for transfer to a target surface without substantial dissociation of the cells. The amnion cell sheet may be an epithelial amnion cell sheet in which at least 60% of cells express epithelial markers identified herein. The amnion cell sheet may be a mesenchymal amnion cell sheet (e.g., mesenchymal amnion stem cell) in which at least 60% of cells express mesenchymal markers identified herein. The amnion cell sheet described herein can be clinical-grade amnion cell sheet meeting predefined specifications for purity, potency, sterility endotoxin levels, and packaged for therapeutic use.

Provided herein also include methods of treating a disease or disorder using the amnion cells described herein. The method can comprise administrating an effective amount of a population of human amnion epithelial cells (e.g., amnion epithelial stem cells), human amnion mesenchymal cells (e.g., amnion mesenchymal stem cells), or both to a subject in need, thereby providing an immunomodulatory or regenerative therapeutic effect in the subject. The disease or disorder can comprise an autoimmune condition or chronic inflammatory disease, fibrotic disorders, neurological and neurodegenerative conditions, respiratory conditions, cardiovascular conditions, musculoskeletal and orthopedic disorders, ocular disorders, dermatologic and would healing, gastrointestinal and hepatic disorders, reproductive and gynecologic conditions, kidney and urinary disorder, and immune-mediated or systemic disorders (e.g., sepsis or systemic inflammatory response syndromes).

In some embodiments, the method can further comprise quantifying expression and/or abundance of immunosuppressive factors such as migration inhibitory factor (MIF), SPP1 and the SPP1 receptor CD44, and selecting amnion-derived cells or an amnion-derived cell sheet that exhibits the immunosuppressive factor level above a predetermined threshold.

Cells can be administered in a manner that permits them to survive, grow, or propagate. Cells may be delivered by injection or implantation. Cells can be administered in doses of from 1×10⁵ to 1×10⁸ cells per kg. In some embodiments, cells are provided in a composition comprising the cells of the present disclosure and at least one pharmaceutically acceptable excipient, carrier or vehicle. Suitable carriers and diluents include, but are not limited to, isotonic saline solutions, for example phosphate-buffered saline. In some embodiments, the composition can be administered by direct injection. The composition can be formulated for parenteral, intramuscular, intravenous, subcutaneous, intraocular, oral, transdermal administration, or injection into the spinal fluid.

In some embodiments, cells are delivered in suspension or embedded in a support matrix such as natural and/or synthetic biodegradable matrices. Natural matrices include, but are not limited to, collagen matrices, fibrin matrices, chitosan, hyaluronic acid, and silk fibroin. Synthetic biodegradable matrices include, but are not limited to, poly(lactic-co-glycolic acid) (PLGA), polyanhydrides, polylactic acid and PEG-based hydrogels. These matrices may provide support for fragile cells in vivo. In some embodiments, the cells are provided in a cell sheet as described above.

Disclosed herein include methods for elucidating the role of a gene in embryo and/or amnion development. In some embodiments, the method comprises obtaining a pluripotent cell where the gene has been modified or knocked out, culturing the pluripotent cell in a culture medium under a condition allowing the pluripotent cell to differentiate into an embryonic structure or embryo like structure, and measuring expression level of one or more amnion gene markers described herein. For example, random and/or targeted mutagenesis can be performed in a pluripotent stem cell and the role of mutated genes in development can be elucidated by using said pluripotent stem cell for generating an embryonic structure, an embryo-like structure, or differentiated cells using the methods described herein and observing any effects using methods known to those of skill in the art. The method can further comprise comparing the measured expression level of the one or more amnion gene markers to a control expression level of the same one or more amnion gene markers in an embryonic structure generated from culturing an unmodified pluripotent cell.

### EXAMPLES

Some aspects of the embodiments discussed above are disclosed in further detail in the following examples, which are not in any way intended to limit the scope of the present disclosure.

### Example 1

### General Experimental Methods

This example describes general experimental methods and data analysis approaches used in Example 2 below.

### Human Amnion Collection

Human amnion tissue samples were collected from healthy pregnant donors after obtaining informed consent and following institutional ethical guidelines. All procedures were approved by the MRC-Wellcome Trust Human Developmental Biology Resource (HDBR) under ethical approval from the London-Fulham Research Ethics Committee (reference: 08/H0712/34+5, IRAS Project ID: 134561). Sample collection followed HDBR standard operating procedures and documentation, including: Patient Information Sheet and Consent Form, version 16; SOP—Recruitment of Donors, version 8; SOP—Collection of Consented Material, version 7; HDBR Background and Protocol, version 10. Tissue samples were obtained from elective caesarean sections or vaginal deliveries, with no known maternal or foetal complications. Detailed covariate information such as age, genotype or medical history of the donors was not available. When the amnion was collected, the yolk sac was readily identifiable as a distinct vascular sac, separate from the embryo, and could be separated from the amnion. The entire amnion was collected without any specific positional preference.

### Tissue Processing

All tissues for sequencing were collected in HypoThermosol FRS preservation solution (H4416-100ML Merck) and stored at 4° C. until processing. Tissue dissociation was conducted within 24 h of tissue retrieval.

Tissues were cut into segments of less than 1 mm³ and washed with RPMI 10% FBS 1% penicillin-streptomycin medium before being digested with trypsin-EDTA 0.25% phenol red (25200072, Thermo Fisher Scientific) for 10-15 min at 37° C. with intermittent shaking. The digested tissue was passed through a 100-μm filter and the cells were collected by centrifugation (500 g for 5 min at 4° C.). Cells were washed with PBS and resuspended in PBS 0.04% BSA before cell counting. In the case of the CS22 amnion sample, after digestion and washing, a reddish cell pellet was observed and red blood cell lysis buffer (eBioscience, 00-4333-57) was used for optimal lysis of erythrocytes in the single-cell suspension.

### 10× Genomics Chromium GEX (Gene Expression) Library Preparation and Sequencing

For the scRNA-seq experiments, cells were loaded according to the manufacturer's protocol for the Chromium Next GEM Single Cell 5 v2 (dual index) kit for the CS17 amnion and Chromium Next GEM Single Cell 3 v3.1 (dual index) kit for the CS16, CS19 and CS22 amnion from 10× Genomics to attain 7,000 cells per reaction. Library preparation was carried out according to the manufacturer's protocol. Libraries were sequenced, aiming at a minimum coverage of 20,000 raw reads per cell, on the Illumina HiSeq4000 or Novaseq 6000 systems using the following sequencing format: read 1, 26 cycles; i7 index, 8 cycles, i5 index, 0 cycles; read 2, 98 cycles.

### 10× Genomics Data Preprocessing

Cell Ranger software from 10× Genomics was used for data preprocessing. Raw sequencing data were organized, with the requirement that sequencing reads be demultiplexed into FASTQ format files for each sample. The tool ‘cellranger mkfastq’ was used to demultiplex raw base call (BCL) files generated by Illumina sequencers into FASTQ files. Reads were mapped to the human reference genome hg38 and counted with GRCh38-3.0.0 annotation using ‘cellranger count’. The data preprocessing workflow was streamlined and standardized to maintain consistency across samples.

### Quality Control

Doublets and maternal cells were removed by the Souporcell software, using the default parameter with ‘--clusters=4’; only ‘singlet’ cells were kept for analysis. Souporcell clusters were shown in the UMAP; cells from a single genotype that clustered into the same Seurat cluster were identified as maternal cells and excluded from the analysis.

To eliminate contamination, the ‘AddModuleScore’ function in Seurat4 package was used to assign scores to cells identified as erythrocytes (markers: HBZ, HBE1, HBG2, HBG1, HBA1, HBA2, HBM, ALAS2, HBB, GYPB, GYPC and GY PA), chorion (markers: CGA, CGB3, GCM1, CGB5, CGB7 and CGB8), yolk sac (markers: AFP, CER1, HHEX, FOXA2 and SPINK1) and blood vessels (markers: CD34, PECAM1, CLDN5, CDH5, ESAM, FLT1 and OGN). Only cells with scores <0 were kept for analysis.

scRNA-seq data were processed using a Seurat4. Initially, each dataset underwent quality control, filtering out cells on the basis of gene expression metrics, specifically retaining cells with gene counts (nFeature_RNA) between 500 and 8,000 and mitochondrial gene content (percent.mt) below 20%.

### Data Integration

The data was normalized using the ‘SCTransform’ method from the Seurat package, using ‘glmGamPoi’ for normalization while regressing out the mitochondrial gene content and cell cycle genes. To mitigate batch effects and integrate data across different sources, 5,000 integration features were selected using Seurat's ‘SelectIntegrationFeatures’ function. ‘PrepSCTIntegration’ function prepared the datasets for integration, and ‘FindIntegrationAnchors’ was used to identify integration anchors, using SCT normalization. ‘IntegrateData’ function, with SCT normalization, was used to integrate the datasets.

### Dimension Reduction and Clustering

Dimensionality reduction and clustering were performed on the integrated scRNA-seq dataset. Principal component analysis was applied using the ‘RunPCA’ function. The ‘RunUMAP’ function was used to generate a UMAP representation, using the first 40 principal components (dims=1:40) and specifying ‘umap-learn’ as the method. A shared nearest-neighbour graph was constructed using ‘FindNeighbors’, again focusing on the first 40 dimensions. The ‘FindClusters’ function was applied with a resolution parameter set to 0.4 to detect distinct cell clusters within the data.

### Identification of Cluster-Specific Marker Genes and Cell Type Annotation

The data was normalized using the ‘NormalizeData’ function and proceeded to scale the data across all genes using the ‘ScaleData’ function. To identify markers for each cluster, the ‘FindAllMarkers’ function was used, focusing only on positive markers (only.pos=TRUE), and setting the minimum percentage of cells expressing the gene (min.pct) at 0.1 and the log fold change threshold (logfc.threshold) at 0.25. The resulting markers were grouped by their respective clusters and sorted to highlight the top markers based on average log fold change.

### GO Enrichment

The Metascape (metascape.org) was utilized for comprehensive gene list annotation and enrichment analysis. GO enrichment analysis was performed across three main categories: biological process (BP), cellular component (CC) and molecular function (MF). For enrichment, we set ‘Min Overlap’=3, ‘P Value Cutoff’=0.01 and ‘Min Enrichment’=1.5.

### Trajectory and Pseudotime Analysis

Trajectory and pseudotime analysis was performed by Monocle3. In brief, a CellDataSet (cds) was prepared from a Seurat object. To integrate UMAP coordinates from Seurat into Monocle3, the UMAP embedding was extracted from cds and aligned with the Seurat UMAP embedding. Trajectory analysis was initiated with ‘learn graph’ and cells ordered based on UMAP trajectories using ‘order_cells’. For identifying genes associated with cell trajectories, ‘graph_test’ was used on the cds, specifying the ‘neighbor graph’ as ‘principal graph’ to highlight genes strongly related to the developmental pathways.

### RNA Velocity

The BAM file was generated using the default parameters of Cell Ranger. For molecule counting, GRCh38 genome annotations from Cell Ranger's prebuilt references were utilized, categorizing the molecules into ‘spliced’, ‘unspliced’ and ‘ambiguous’. Repeat annotation files were acquired from the UCSC Genome Browser. AnnData objects (h5ad) were built with ScanPy and ScVelo. RNA velocity analysis was performed by the scVelo package in Python. In brief, the data was filtered and normalized using ‘scv.pp.filter_and_normalize’, setting a minimum threshold of 30 shared counts and selecting the top 2,000 highly variable genes to focus on the most informative aspects of the dataset. The neighbourhood moments were calculated with ‘scv.pp.moments’, using 30 principal components and 30 nearest neighbours, to capture the local structure and variability within the data. The RNA velocity was estimated using the stochastic model through ‘scv.tl.velocity’. The velocity graph was constructed using ‘scv.tl.velocity_graph’ to visualize.

### Cell-Cell Communication

CellChat R package was used to analyze cell-cell communication networks based on the scRNA-seq dataset. A CellChat object was created by loading the normalized gene expression data into the CellChat environment, followed by assigning cell identities based on their respective metadata. The human ligand-receptor interaction database (CellChatDB.human) was used for the analysis, focusing specifically on ‘Secreted Signaling’ pathways to tailor our investigation towards secretome-mediated interactions. The communication probabilities were computed to infer the cellular communication networks. Communications between cell groups with fewer than ten cells were filtered out.

### Diffusion Map

Diffusion map analysis was performed using the Destiny package, considering the 2,000 most variable protein-coding genes. ‘Diffusion map 1’ was set as pseudotime.

### Human, Monkey and In Vitro Model Data Comparison

Human CS7 embryo data were downloaded from Array Express (E-MTAB-9388). Cells annotated as epiblast, amnion, hypoblast and non-neural ectoderm were selected for the analysis. Monkey CS8_CS11 data were obtained from Gene Expression Omnibus (GEO) under accession number GSE193007. Cells annotated as amnion, ectoderm, epiblast, extra-embryonic mesoderm, mesenchyme, surface ectoderm1, surface ectoderm2 and visceral endoderm were selected for the analysis. Stem cell-derived model data were downloaded from NCBI Gene Expression Omnibus (GEO): GSE179309, GSE205611 and GSE134571. Those datasets were integrated by the Seurat function ‘merge’.

### Immunostaining

Human amnion sections were fixed using 4% paraformaldehyde and permeabilized with 0.1% Triton X-100. Non-specific binding was blocked using 10% donkey serum. The sections were incubated with primary antibodies, followed by washing and incubation with fluorophore-conjugated secondary antibodies. After additional washes, nuclear counterstaining was performed using DAPI. The samples were mounted with anti-fade medium and visualized under a confocal microscope.

### Human iPS Cell Culture

The human iPS cell line (WTC-11) was generously provided by Dr Bruce R. Conklin (Gladstone Institute of Cardiovascular Disease, UCSF). Human naive iPS cells were cultured on Matrigel-coated plates in 4CL medium (1:1 mix of Neurobasal medium (Gibco, 21103049) and Advanced DMEM/F12 (Gibco, 12634028) supplemented with N₂ (Gibco, 17502048) and B27 (Gibco, 17504044), sodium pyruvate (Corning, 25000CL), non-essential amino acids (Corning, 25025CL), GlutaMAX (Gibco, 35050061), penicillin-streptomycin (HyClone, SV30010), 10 nM DZNep (Selleck, S7120), 5 nM TSA (Vetec, V900931), 1 μM PD0325901 (Axon, 1408), 5 μM IWR-1 (Sigma, I0161), 20 ng ml-1 human LIF (Peprotech, 300-05), 20 ng ml-1 activin A (Peprotech, 120-14E), 50 μg ml⁻¹ L-ascorbic acid (Sigma, A8960) and 0.2% (v/v) Matrigel, Cells in 4CL were cultured at 37° C., 5% O₂ and 5% CO₂. To induce specific signaling pathways, the medium was supplemented with 20 ng ml⁻¹ MDK (PeproTech, 450-16), 100 nM RA (STEMCELL Technologies, 72262) and 20 ng ml⁻¹ BMP4 (PeproTech, 120-05ET). These cells were maintained in culture and collected for analysis on the fifth day.

Quantitative PCR (qPCR)

Total RNA was extracted using the Direct-zol RNA Purification Kit, Miniprep (Zymo Research, R2051), according to the manufacturer's protocol. RNA concentration and purity were assessed using a Nanodrop spectrophotometer. For qPCR, 10 ng of RNA per reaction was used with the Luna Universal One-Step RT-qPCR Kit (New England Biolabs, E3005L) Reactions were carried out under the following cycling conditions: reverse transcription at 55° C. for 10 min, initial denaturation at 95° C. for 1 min, followed by 40 cycles of 95° C. for 10 s and 60° C. for 30 s. Relative gene expression was calculated using the ΔΔCt method, with GAPDH as the internal control. Primers were synthesized by Integrated DNA Technologies (IDT).

### Statistics and Reproducibility

Immunofluorescence staining experiments were repeated independently with consistent results. No statistical method was used to predetermine sample size. For scRNA-seq experiments, the number of samples was determined by tissue availability, which also guided collection and processing. Randomization was not applied during data collection or processing.

Putative maternal cells were excluded on the basis of the expression of maternal markers and genotype mismatches. This exclusion was performed before downstream analyses. Investigators were not blinded to group allocation during experiments or outcome assessment.

Statistical analyses were conducted using GraphPad Prism (v8.2.0). Data distribution was assumed to be normal, although this was not formally tested. For scRNA-seq analyses, differentially expressed marker genes were identified using Seurat (v4.3.0) with a two-sided Wilcoxon rank-sum test. Details regarding sample sizes, statistical tests and P values are provided in the main text, figures, figure legends and supplementary tables.

### Data Availability

Sequencing data that support the findings of this study have been deposited in the Gene Expression Omnibus (GEO) under accession number GSE260715. Previously published human CS7 embryo data were downloaded from Array Express (E-MTAB-9388). Monkey CS8_CS11 data were obtained from GEO under accession number GSE193007. Stem cell-derived model data were downloaded from GSE179309, GSE205611 and GSE134571. The human reference genome (GRCh38/hg38) used for alignment was downloaded from the 10× Genomics website (cf.10×genomics.com/supp/cell-exp/refdata-gex-GRCh38-2020-A.tar.gz).

### Example 2

### Single Cell Analysis of Human Amnion

The amnion is a critical extra-embryonic structure that supports fetal development, yet its ontogeny remains poorly defined. This example describes using scRNA-seq to profile various cell types present in human amnion during the first trimester of human pregnancy to gain insight into their interactions and potential functional contributions.

Using single-cell transcriptomics, major cell types and subtypes were identified in the human amnion across the first trimester of pregnancy, broadly categorized into epithelial, mesenchymal and macrophage lineages. Epithelial-mesenchymal and epithelial-immune transitions were uncovered, highlighting dynamic remodelling during early pregnancy. The results presented herein further revealed key intercellular communication pathways, including BMP4 signaling from mesenchymal to epithelial cells and TGF-β signaling from macrophages to mesenchymal cells, suggesting coordinated interactions that drive amnion morphogenesis. In addition, integrative comparisons across humans, non-human primates and in vitro stem cell-based models reveal that stem cell-based models recapitulate various stages of amnion development, emphasizing the need for careful selection of model systems to accurately recapitulate in vivo amnion formation. Collectively, the findings presented herein provide a detailed view of amnion cellular composition and interactions, advancing the understanding of its developmental role and regenerative potential.

### Cell Composition of Human Amnion in the First Trimester

To explore the dynamics of transcriptional changes during amnion development, seven human amnion samples were collected, representing 5-9 weeks of pregnancy and corresponding to Carnegie stages (CS) 16, 17, 19, 22 and 23, respectively. CS16 embryos have developed limb buds, the otic vesicle, early eye structures and the primitive heart tube, along with forming somites and the neural tube. CS17 embryos have developed hand rays, cartilage, ribs, intercostal muscles, mammary glands and the thymus. By CS19, embryos have developed the cerebral aqueduct, middle cerebral artery, renal artery and tibia. By CS22, the embryonic brain has developed nerve cell clusters and bundles of nerve fibres, and ossification has begun in the clavicle and long bones (FIG. 1, panels a,b).

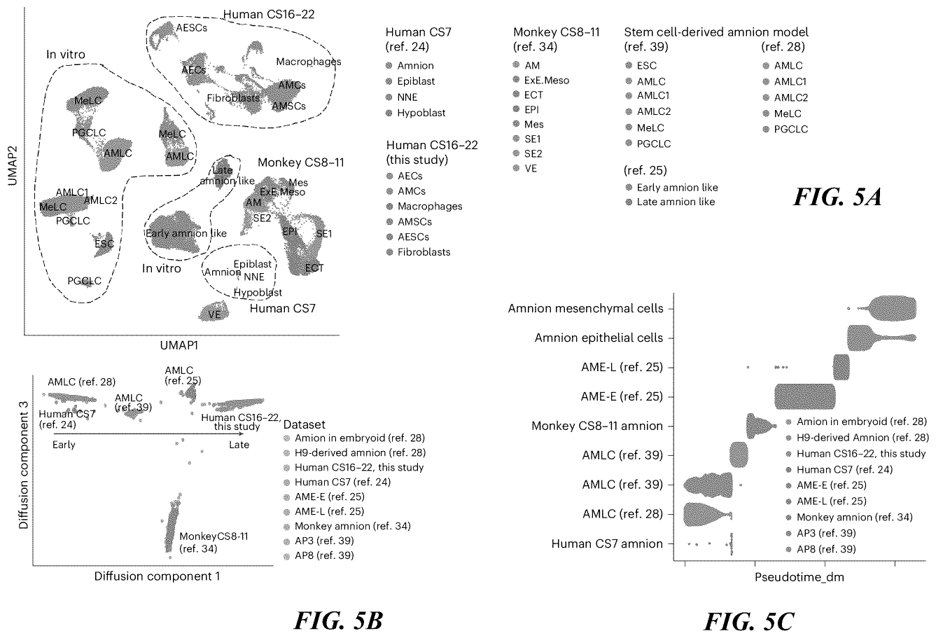

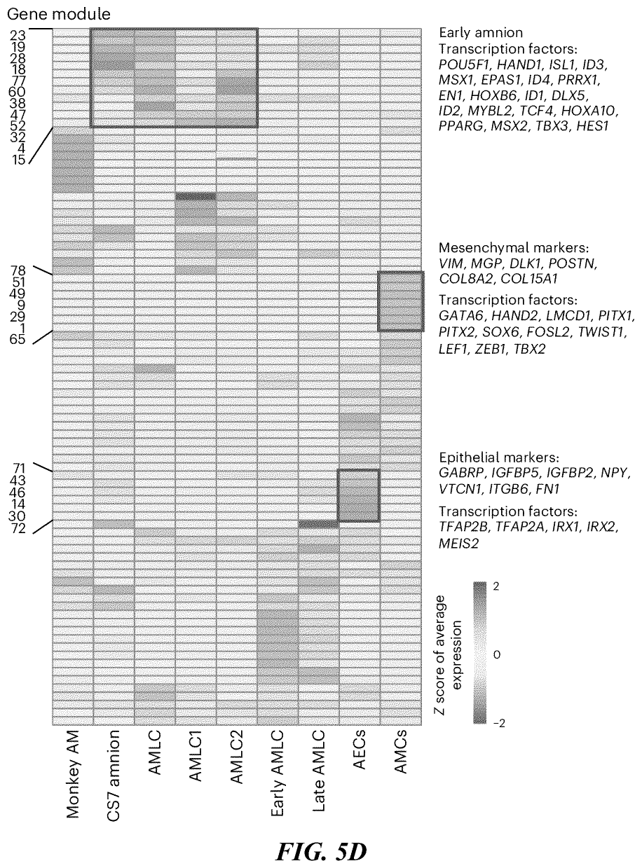

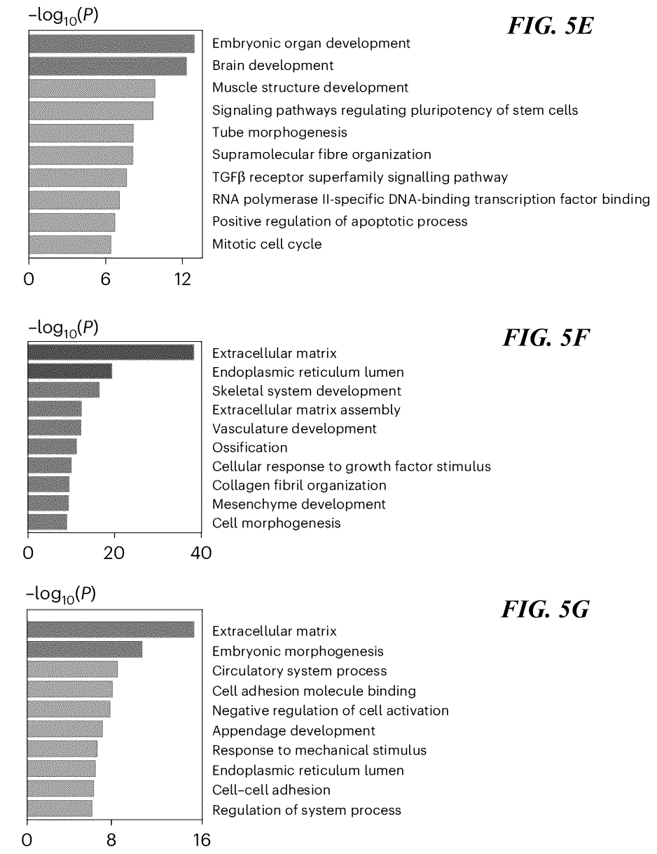

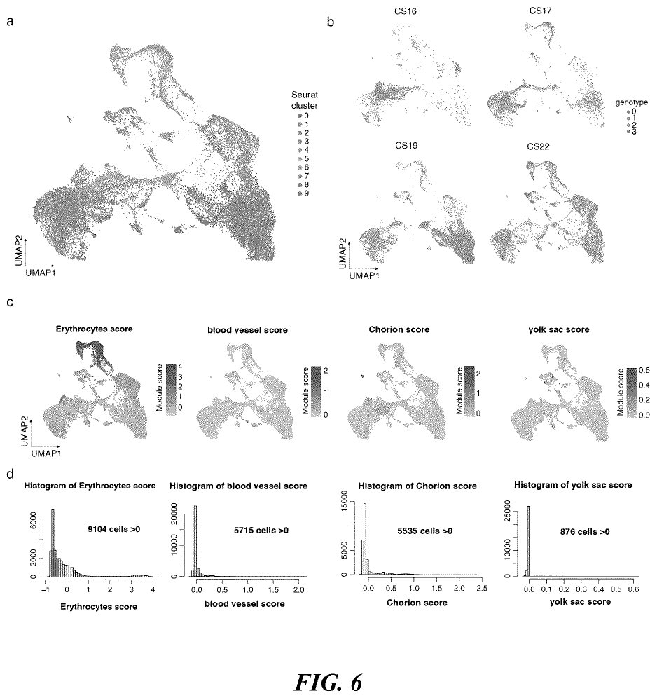

Single-cell suspensions were prepared from four of these samples (CS16, CS17, CS19 and CS22) and performed scRNA-seq using the 10× Genomics Chromium system (Extended Data FIG. 1, panel a). Cells with fewer than 500 or more than 8,000 genes expressed were excluded. In addition, cells with more than 20% mitochondrial reads were excluded to remove dead cells. Cell doublets were removed by Souporcell analysis. In addition, amnion tissue can be contaminated during dissection with maternal cells such as maternal blood cells and blood vessels, Souporcell was used to analyze and cells that could be of maternal origin were removed (FIG. 6, panel a). The cells were also scored and those with marker gene expression characteristic of yolk sac, chorion, blood vessels and erythroid cells were excluded (FIG. 5, panels c, d). In total, 14,027 single cells passed quality control and were included in the analysis. Data from the four stages were integrated using the ‘IntegrateData’ function in Seurat4.

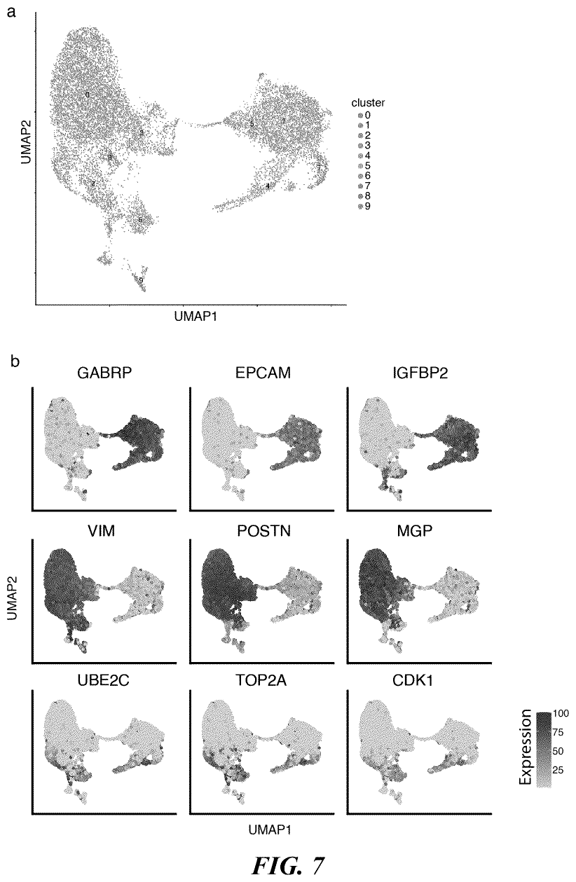

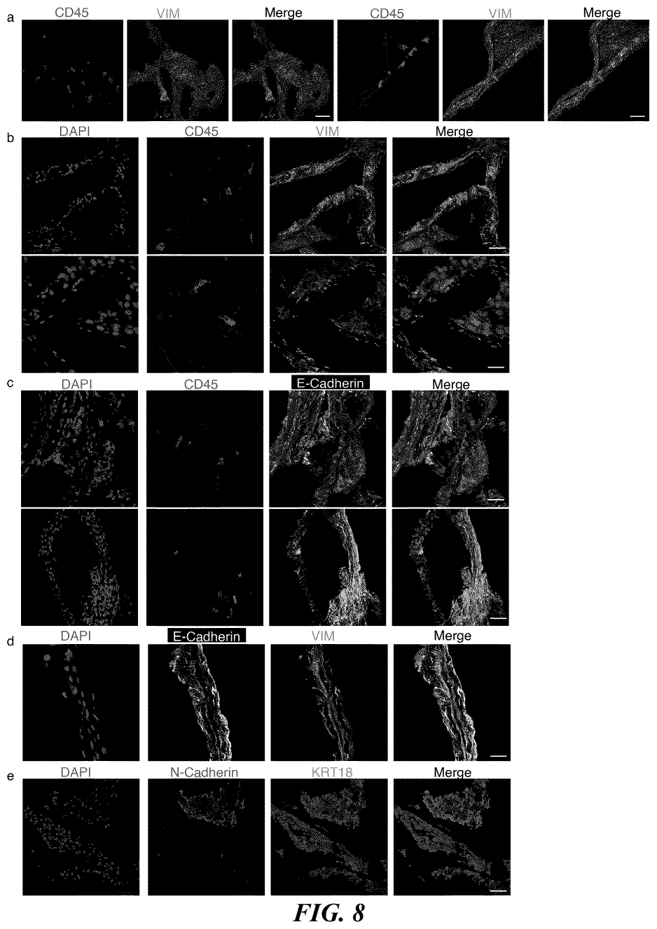

Unsupervised clustering utilizing the Seurat package revealed ten distinct cell clusters defined by their transcriptional signatures (FIG. 7, panel a). A total of six major cell types were identified among the ten clusters based on their marker genes (FIG. 1, panels c, d). These include amnion epithelial cells (AECs, clusters 1 and 5, marked by GABRP and KRT18), amnion mesenchymal cells (AMCs, cluster 0, marked by MGP and VIM), fibroblasts (cluster 3, marked by COL6A1 and COL5A1), macrophages (cluster 9, marked by MRC1 and CD36) and two clusters of actively proliferating cells (marked by CDK1 and TOP2A), which were defined as amnion mesenchymal stem cells (AMSCs, clusters 2, 6 and 8) and amnion epithelial stem cells (AESCs, clusters 4 and 7) based on the expression of lineage-specific genes (FIG. 1, panel d and FIG. 7, panel b). Their stem cell characteristics were also demonstrated by subsequent pseudotime analysis. The immunofluorescence staining of sectioned tissues confirmed the presence of epithelial cells (expressing E-Cadherin and KRT18), mesenchymal cells (expressing VIM), fibroblasts (expressing N-Cadherin), and macrophages (expressing CD45) in human CS19 and CS23 amnion tissues (FIG. 1, panel e and FIG. 8, panels a-e).

### Cell Subtypes and Lineage Trajectories in Amnion Development

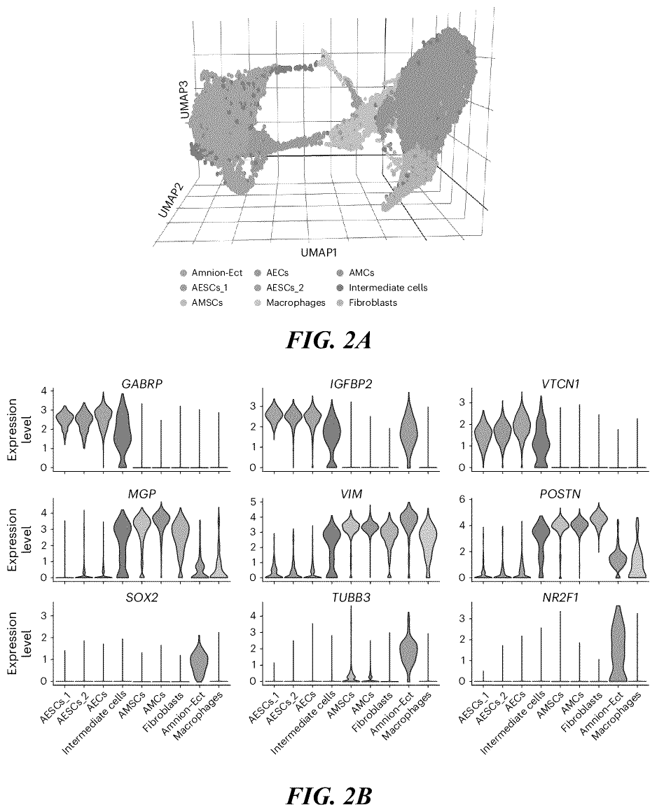

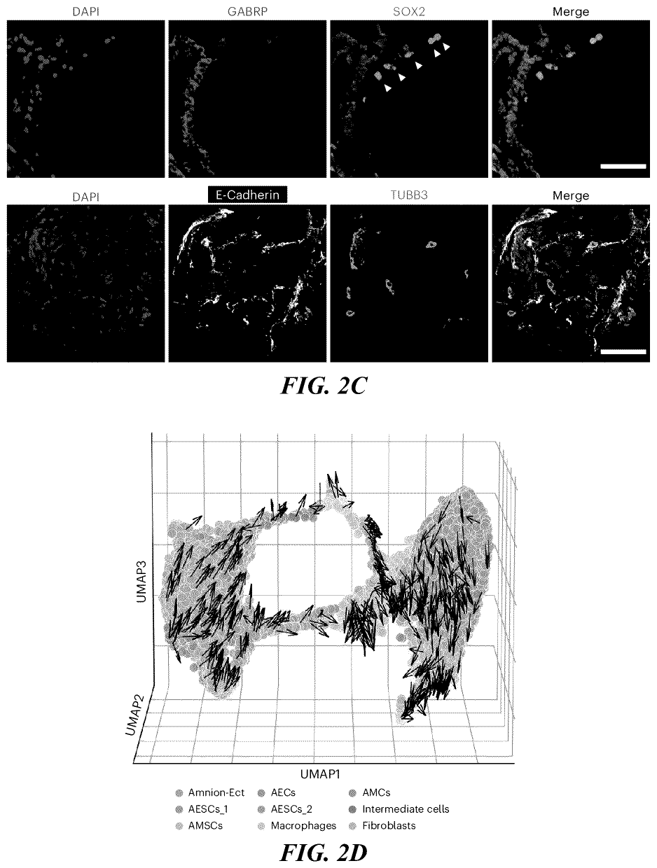

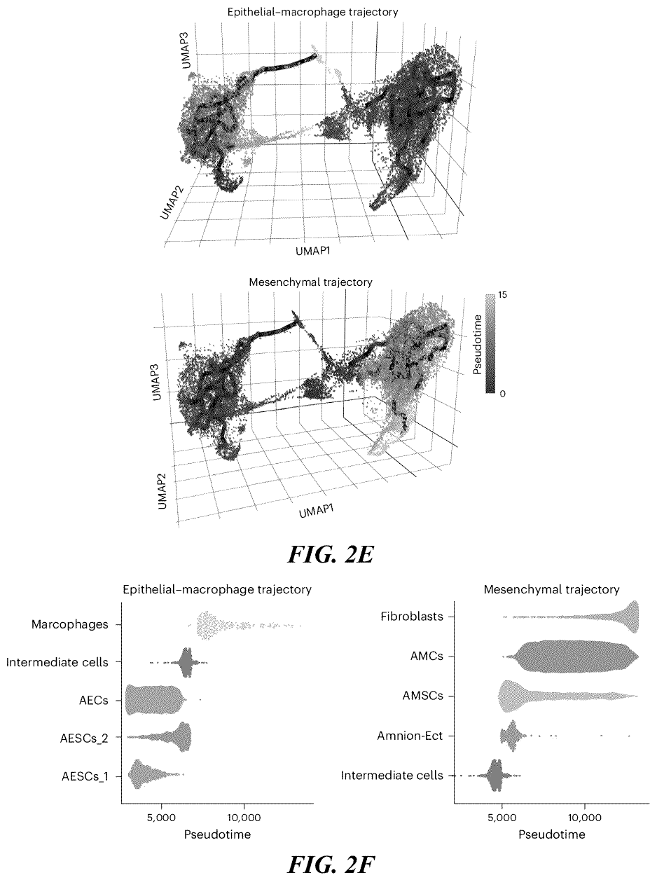

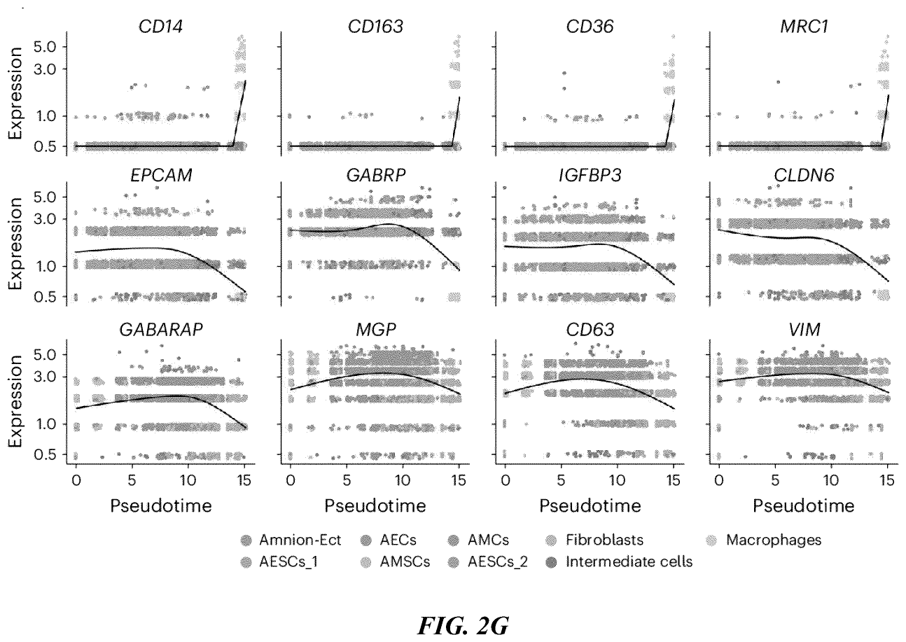

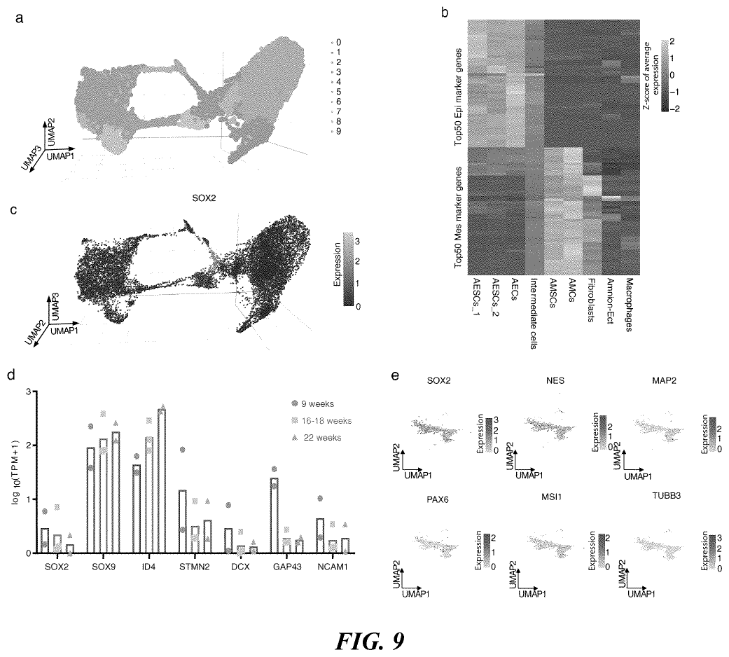

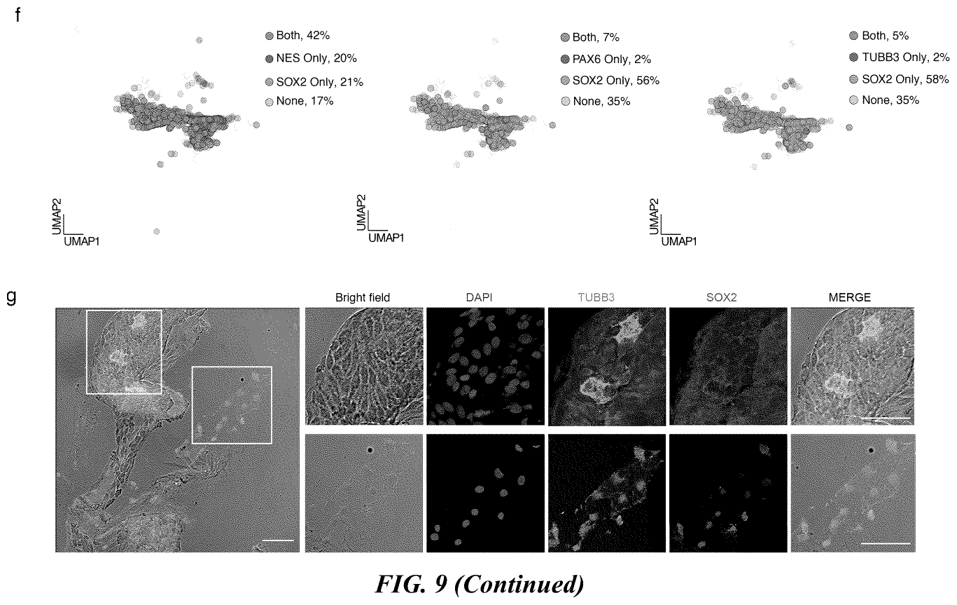

To provide a more comprehensive and detailed depiction of the amnion's cellular composition, three-dimensional (3D) Uniform Manifold Approximation and Projection (UMAP) plots were used (FIG. 2A and FIG. 9, panel a). This approach allowed us to further subdivide the AESCs into two distinct groups, labelled as AESCs_1 (cluster 7) and AESCs 2 (cluster 4). Moreover, a population of intermediate-state cells (cluster 5) that express both epithelial and mesenchymal marker genes was identified (FIG. 2B and FIG. 9, panel b). In addition, the analyses revealed a group of cells with high expression of ectoderm markers such as SOX2, TUBB3 and NR2F1 (FIG. 2B and FIG. 9, panel c), which were labelled as amnion ectodermal cells (Amnion-Ect, AM-Ect). These ectoderm markers were also found to be expressed in the bulk RNA-seq of first and second trimester of human amnion samples (FIG. 9, panel d) and in the scRNA-seq of CS8-11 cynomolgus monkey amnion cells (FIG. 9, panels e, f). Immunofluorescence staining suggests the presence of amnion ectodermal cells (expressing SOX2 and TUBB3) in the CS16 and CS19 amnion section (FIG. 2C and FIG. 9, panel g).

To investigate the developmental trajectories within amnion cells, three pseudotime analysis methods were utilized. RNA velocity revealed two primary trajectories: epithelial and mesenchymal. The epithelial trajectory further branched into two distinct paths: one transitioning from AESCs 1 to AESCs_2 and the other from AESCs 1 to AECs and then to macrophages. The mesenchymal trajectory delineated a progression from AMSCs to AMCs and finally to fibroblasts (FIG. 2D). Similarly to the RNA velocity results, trajectory and pseudotime analyses using Monocle3 and Destiny also revealed the same two developmental trajectories (FIGS. 2E and 2F).

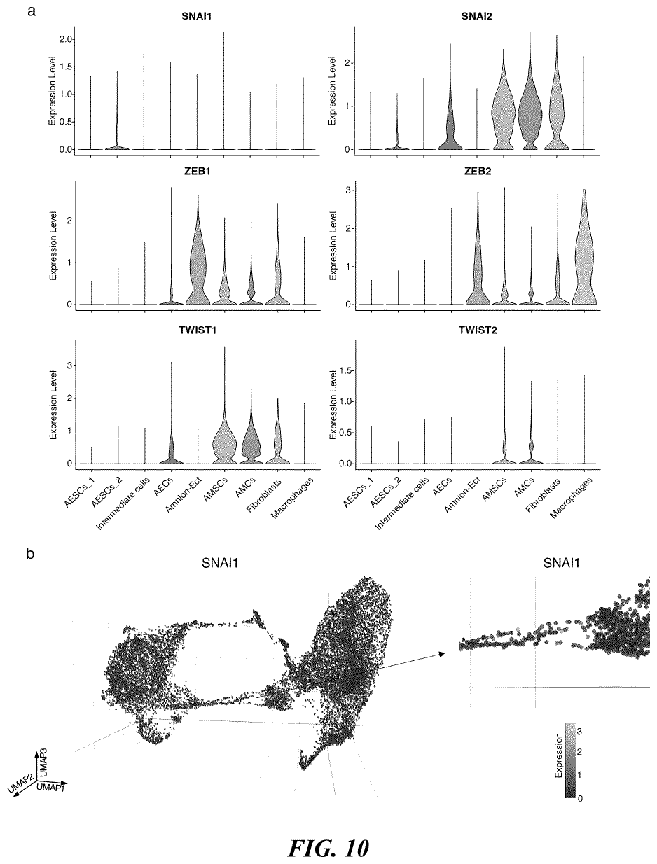

Based on the UMAP and pseudotime analyses, AESCs_2 and intermediate cells may serve as bridges between epithelial and mesenchymal lineages, suggesting that an EMT occurs in the amnion. Consequently, the expression of transcription factors related to EMT in different cell subtypes was examined. The analyses indicated that most EMT-related transcription factors were highly expressed in the mesenchymal lineage (FIG. 10, panel a). Specifically, expression of SNAI1 and SNAI2 was detected in the AESCs 2 cells, while expression of SNAI2, ZEBI and TWIST1 was observed in the intermediate cells. Interestingly, high SNAI1 expression was discovered only in the AESCs 2 cells that were closest to the mesenchymal lineage (FIG. 10, panel b), suggesting that AESCs_2 might be transitioning into mesenchymal cells through EMT.

The analyses also identified transcripts that change expression in accordance with pseudotime. Specifically, along the epithelial-macrophage trajectory, a progressive increase was observed in the expression levels of epithelial and macrophage marker genes, such as GABRP, IGFBP3, MRC1 and CD36. By contrast, mesenchymal marker genes such as MGP and VIM are systematically downregulated (FIG. 2G). Interestingly, this trend was reversed in the mesenchymal trajectory, where mesenchymal marker genes showed an increase in expression, highlighting the distinct and dynamic cellular behaviors in different developmental paths.

### Intercellular Communication in Amnion Development

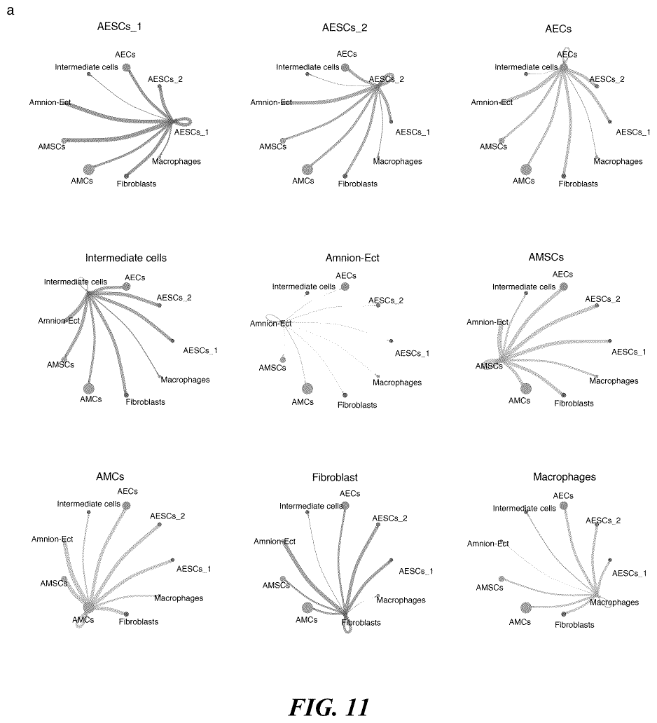

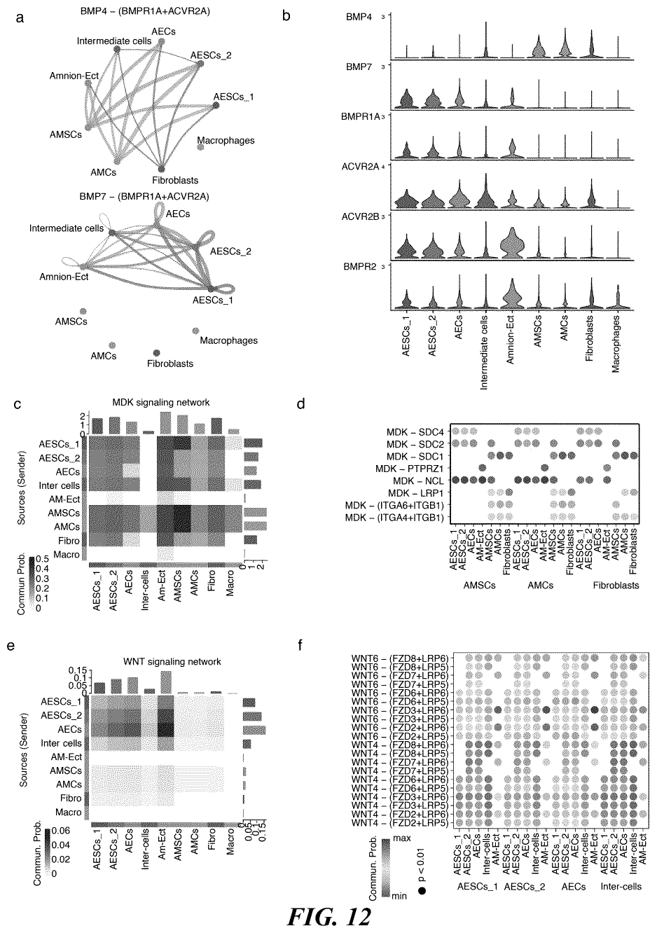

To investigate intercellular communication among amniotic cells, CellChat was utilized, which uncovered numerous potential interactions between various cell populations (FIGS. 3A and 3B and FIG. 11, panel a). The expression of receptors and ligands within cells was examined to identify the roles of different cell types in the interaction network. It was found that macrophages and intermediate cells exhibited an outgoing profile, primarily expressing ligands, whereas amnion ectodermal cells displayed an incoming profile, predominantly expressing receptors. Other cell types demonstrated a combination of both outgoing and incoming signaling capabilities (FIG. 3C).

Cells were classified into three patterns based on their expression of genes encoding secreted signaling ligands (FIGS. 3D and 3E). The epithelial pattern included AESCs_1, AESCs 2, AECs and intermediate cells, which expressed ligands associated with bone morphogenetic protein (BMP), Wingless/Integrated (WNT), platelet-derived growth factor (PDGF) and growth differentiation factor (GDF) signaling. The mesenchymal pattern consisted of AMSCs, AMCs and fibroblasts, which expressed ligands associated with midkine (MDK), non-canonical Wnt (ncWNT), hepatocyte growth factor (HGF) and insulin-like growth factor (IGF). Finally, the macrophage pattern showed expression of secreted phosphoprotein 1 (SPP1) and transforming growth factor beta (TFG-β).

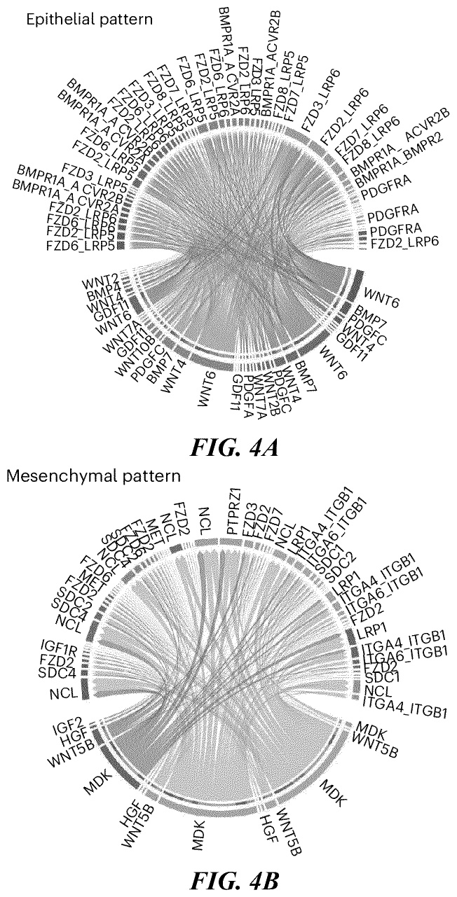

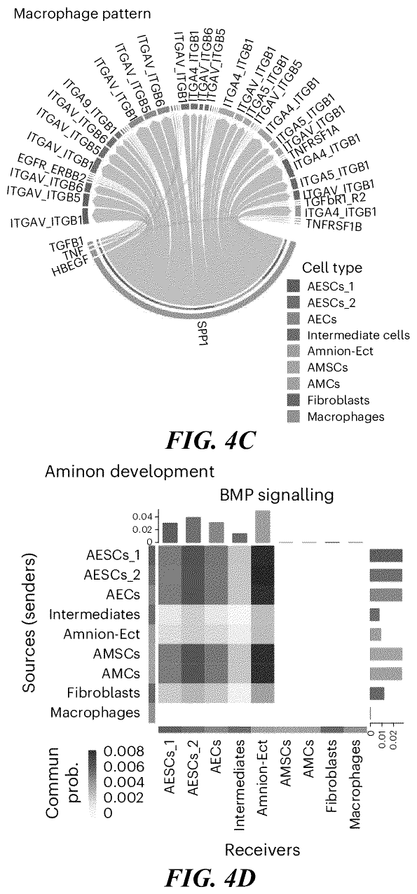

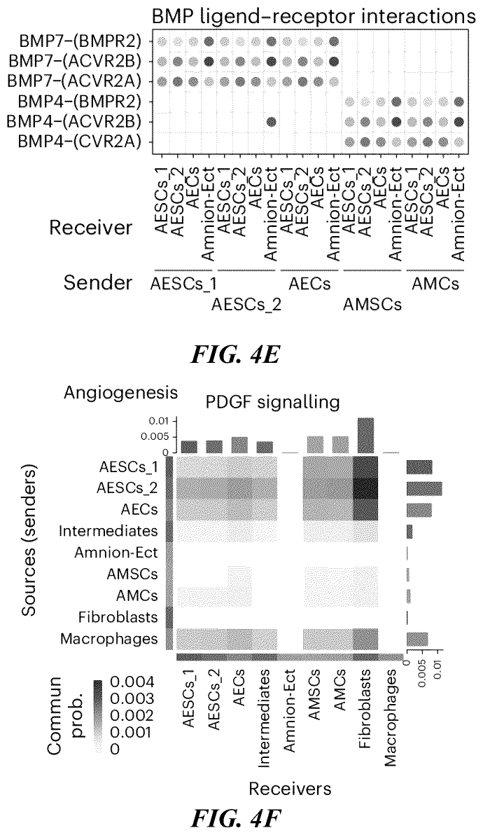

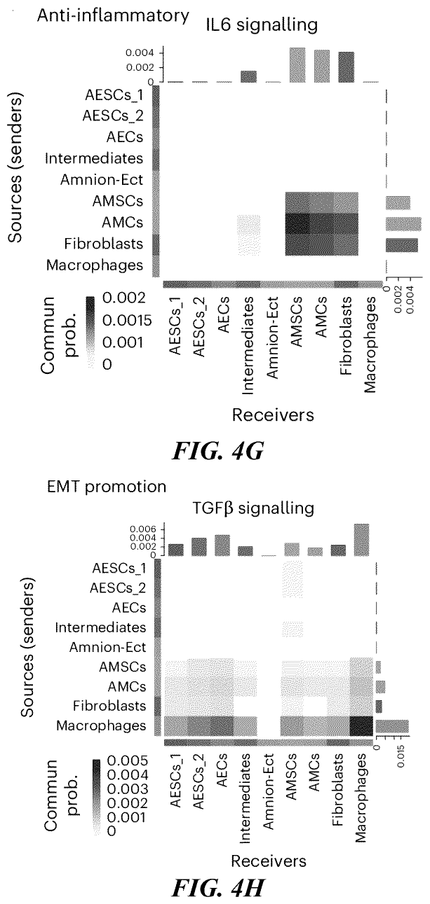

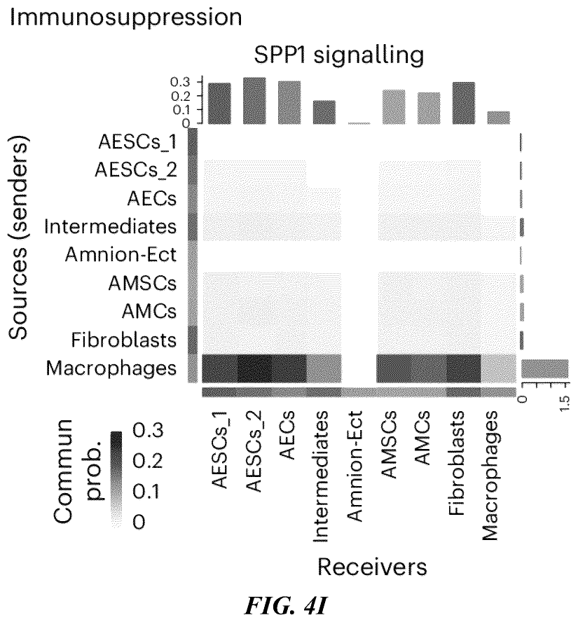

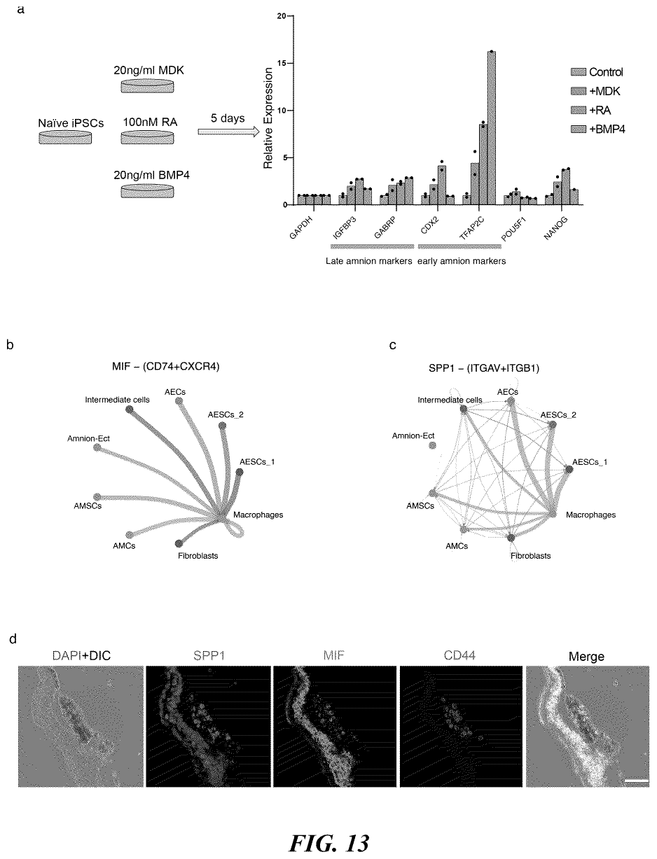

The analyses identified several growth factors that are linked to specific growth and developmental stages of amniotic cells, illustrating a complex intercellular communication network within the amnion. By analyzing ligand and receptor expression, Applicant was able to identify the likely signaling and responding cells across different cell populations (FIGS. 4A-4C). Specifically, the analyses indicated that within the BMP signaling pathway, which is known to be critical for amnion development, cells of the epithelial lineage function primarily as recipients of BMP signals (FIG. 4D). Interestingly, it was found that BMP4 was primarily expressed by amnion mesenchymal lineage, whereas BMP7 was predominantly expressed by the epithelial cells themselves (FIG. 4E and FIG. 12, panel a). The receptors for these proteins, BMPR1A, ACVR2A, ACVR2B and BMPR2, were mainly expressed in the epithelial lineage cells (FIG. 12, panel b). In addition, potential crosstalk between the MDK and WNT signaling pathways in amnion cells was also identified (FIG. 12, panels c-f). In the in vitro human stem cell differentiation experiments, BMP4 treatment of induced pluripotent stem (iPS) cells resulted in the upregulation of both early and late amnion markers (FIG. 13, panel a).

Some signaling pathways were also related to the characteristics of the amnion, including angiogenesis (PDGF, FIG. 4F), anti-inflammatory effects (IL6, FIG. 4G), EMT promotion (TGF-β, FIG. 4H) and immunosuppression (SPP1, FIG. 4I). The immunomodulatory properties of the amnion were particularly striking as amnion cells expressed various immunosuppressive factors, such as macrophage migration inhibitory factor (MIF) (FIG. 13, panel b) and SPP1 (FIG. 13, panel c), which play crucial roles in inhibiting immune responses. Immunostaining of CS16 amnion sections confirmed the expression of MIF, SPP1 and its receptor CD44 in the amnion (FIG. 13, panel d). The expression of immunosuppressive factors provides a possible explanation for the amnion's capacity to inhibit immune responses. Overall, these pathways highlight the complexity of cellular communication and may play an integral role in coordinating cellular interactions during amnion development.

### Analysis of Amnion Development In Vivo and In Vitro

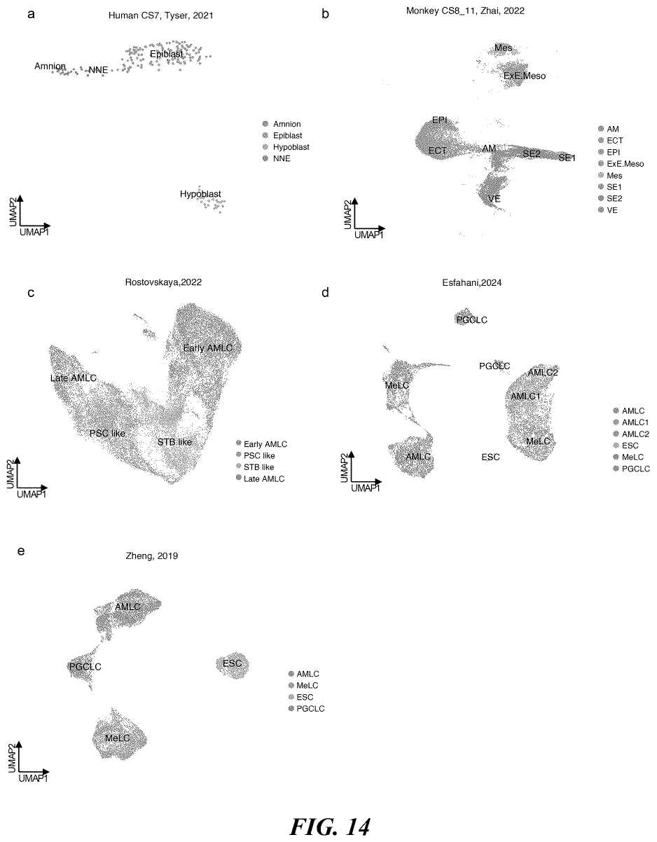

To investigate the developmental dynamics of amnion across different species and experimental conditions, published scRNA-seq datasets from human CS7 (FIG. 14, panel a) were combined with data from monkey CS8-11 (FIG. 14, panel b). In addition, three sets of in vitro-derived amnion-like cells generated from stem cells were incorporated (FIG. 14, panels c-e) and data from our own study on human amnion samples CS16-22 was included.

By leveraging a combined UMAP analysis, a clear developmental progression of amnion formation was identified (FIG. 5A). To further delineate this trajectory, amnion cells were isolated from these datasets and a diffusion map analysis was performed (FIG. 5B). Interestingly, the present findings revealed that the different in vitro amnion models correspond different developmental stages in vivo. Specifically, the amnion models derived from human pluripotent stem (hPS) cells using either in a microfluidic device (Nature, 2019 September; 573 (7774): 421-425. doi: 10.1038/s41586-019-1535-2) or 3D biomimetic culturing (Nat Commun, 2024 Jan. 2; 15 (1): 167. doi: 10.1038/s41467-023-43871-2) resembled earlier amnion stages (around CS7). By contrast, amnion-like cells derived from two-dimensional (2D) hPS cell cultures (Cell Stem Cell, 2022 May 5; 29 (5): 744-759.e6. doi: 10.1016/j.stem.2022.03.014) more closely reflected later stages of amnion development (CS11-16) (FIG. 5C). This divergence highlights variations in developmental timing across different in vitro models, emphasizing the need for careful selection of model systems to accurately recapitulate in vivo amnion development.

Building on this integrative analysis, differential expression and gene module analyses were performed along the amnion developmental trajectory. Genes were categorized into distinct modules based on their expression patterns, and their average expression was visualized in a heatmap (FIG. 5D). Several transcription factors were identified, including POU5F1, HAND1 and ISL1, as being enriched in the early stages of amnion development. Gene Ontology (GO) annotations for these genes were predominantly associated with pathways such as ‘embryonic organ development’ and ‘signalling pathways regulating pluripotency of stem cells’ (FIG. 5E). By contrast, late-stage AMCs predominantly express mesenchymal markers such as COL15A1 and MGP, along with transcription factors related to EMT, including GATA6, TWIST1 and ZEB1. GO annotations for these genes were enriched in ‘extracellular matrix’ and ‘mesenchyme development’ (FIG. 5F). In late-stage AECs, gene expression was enriched for epithelial markers such as GABRP and IGFBP5, as well as transcription factors TFAP2A and TFAP2B. GO annotations for these genes were associated with ‘extracellular matrix’ and ‘cell adhesion molecule binding’ pathways (FIG. 5G). This comprehensive genomic analysis illustrates the dynamic changes that occur throughout amnion development.

### Discussion

Single-cell analysis of the human amnion presented herein has revealed a dynamic cellular landscape, developmental trajectories and intercellular interactions between different amnion cell types. Within the first trimester amnion, six major cell types and nine cell subtypes spanning epithelial, mesenchymal and macrophage lineages were identified. In addition, a population of amnion ectodermal cells expressing some neural-related genes was also discovered.

Although traditionally considered a non-neuronal tissue, the amnion has been reported to contain mesenchymal cells with neural progenitor-like characteristic and neurotransmitter metabolism capabilities. Furthermore, neural-related genes such as SOX9, ID4 and STMN2 have been detected in human amnion samples from both the first and second trimester. The results presented herein further show that SOX2-positive ectodermal cells also express neural markers such as TUBB3 and NR2F1, suggesting that these neural progenitor-like cells in the amnion are probably ectodermal cells. However, higher-resolution immunofluorescence imaging would be required to confirm the presence of SOX2-positive cells and to refine our understanding of their morphology and spatial arrangement.

In this study, the entire amnion was collected without specific positional selection, and potential regional variations within the tissue were not specifically addressed. This limitation may contribute to the observed variability. Future studies using spatial transcriptomics or other positional mapping techniques could provide a deeper insight into the spatial heterogeneity and regional distinctions within the amnion.

The present study further demonstrates the potential developmental pathways of epithelial, mesenchymal and macrophage lineages in the human amnion. The observed EMT and epithelial-to-immune transitions (EIT) suggest dynamic cellular remodelling and immune modulation as the amnion develops during the first trimester of human pregnancy. These findings align with previous studies indicating the presence of EMT and EIT in the amnion and their importance in amniotic membrane remodelling. However, further research will be necessary to provide deeper insights into these processes and their functional implications.

Beyond working as a protective barrier, the amnion is a major source of several growth factors crucial for embryogenesis, including EGF, FGF, PDGF and VEGF, which are involved in angiogenesis, tissue repair and immunomodulation. However, the specific cell types that secrete these growth factors remained unclear. Leveraging scRNA-seq data, three distinct secretion patterns were identified within the amnion: epithelial (MIF, WNT, BMP, GDF, PDGF and activin), mesenchymal (MDK, ncWNT, HGF, IGF and IL6) and macrophage (SPP1, TGF-β and CCL). Previous studies have shown that BMP4 promotes amnion development, and the recent work confirmed that BMP4 is essential for the epiblast differentiation into amnion in a stem cell-derived human embryo model. Consistent with these results, it is also shown here that BMP4 treatment of iPS cells led to the upregulation of both early and late amnion marker expression. The present findings reveal that immune cells within the amnion express TGF-β, a cytokine known to promote EMT. This result suggests a potential mechanism in which macrophages may facilitate EMT via TGF-β secretion, thereby enhancing tissue repair. Moreover, it was observed that amnion cells express immunosuppressive factors such as SPP1, MIF and TGF-β, which are known to inhibit immune responses. These findings further support the immunosuppressive properties of the amnion when used as a clinical biomaterial, highlighting its potential to modulate the immune environment and reduce inflammation during tissue repair. Clinically, this understanding can be leveraged to enhance wound healing, reduce inflammation and promote tissue regeneration in applications such as treating burns, chronic wounds and organ injuries, demonstrating the amnion's promise in regenerative medicine.

The comparative analysis of amnion RNA-seq data from human, non-human primate and in vitro amnion models indicates that stem cell-derived amnion models correspond to various stages of in vivo amnion development. Specifically, the amnion models derived from hPS cells using a microfluidic device and 3D biomimetic culture appear to reflect earlier developmental stages compared with amnion-like cells derived from 2D culturing of hPS cells. This difference might be linked to their respective culturing methods: microfluidic and 3D biomimetic culture systems more closely replicate the complex, dynamic conditions of the embryonic environment by providing a 3D, fluid-based context that supports cell-cell and cell-matrix interactions. This supports more accurate tissue development and spatial organization, resembling early stages of amnion development. By contrast, 2D culture systems tend to mimic a more mature structure of the amnion membrane by promoting flattened, layered cell growth, which may better reflect the architecture of foetal membranes. In addition, the present study identified TFAP2A and TFAP2B as key transcription factors enriched in the development of AECs, while GATA6, HAND2 and SOX6 were highly expressed in AMCs. The expression patterns of these transcription factors suggest their involvement in regulatory pathways governing amnion development. However, their precise roles remain to be explored in future studies.

In conclusion, the findings presented herein provide comprehensive insights into the complex cellular architecture of amnion, highlighting its potential roles in embryonic development and tissue repair. This cellular map serves as a valuable resource for future functional studies on amnion development, in vitro amnion models and potential therapeutic applications.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1.-14. (canceled)

15. A method of detecting perturbation-induced changes in amnion cells, comprising:
introducing a perturbation to an amnion cell model or an isolated amnion cell population comprising cells selected from the group consisting of: amnion epithelial cells, amnion epithelial cells, fibroblasts, macrophages, amnion mesenchymal stem cells, and amnion epithelial stem cells; and
determining a perturbation-induced change in one or more gene signatures or pathways of the amnion cell model or the isolated amnion cell population, and
evaluating the effect of the perturbation on the amnion cell model or the isolated amnion cell population by detecting expression of one or more amnion epithelial cell markers, amnion mesenchymal cell markers, fibroblast markers, macrophage markers, amnion mesenchymal stem cell markers, and/or amnion epithelial stem cell markers.

16. The method of claim 15, wherein the perturbation is a physical condition or a chemical condition.

17. The method of claim 15, wherein the perturbation comprises a genetic perturbation, a drug candidate, an absence or presence of a test agent, an increase or decrease of temperature, light, pressure, pH value, or a combination thereof.

18. The method of claim 15, wherein the perturbation comprises an agent selected from the group consisting of: cytokines, transcription factors, signaling molecules, or a combination thereof.

19. The method of claim 15, wherein introducing the perturbation to the amnion cell model or the isolated amnion cell population comprises contacting the amnion cell model or the isolated amnion cell population with the perturbation in a culture medium or culturing the amnion cell model or the isolated amnion cell population in a culture medium in the presence of the perturbation.

20. The method of claim 15, wherein introducing the perturbation to the amnion cell model or the isolated amnion cell population comprises performing RNA interference or CRISPR-Cas gene editing.

21. The method of claim 15, wherein determining the perturbation-induced change in the one or more gene signatures or pathways comprises determining the expression level of the one or more amnion epithelial cell markers, amnion mesenchymal cell markers, or both.

22. The method of claim 15, wherein determining the perturbation-induced change in the one or more gene signatures or pathways comprises comparing the one or more gene signatures or pathways of the amnion cell model or isolated amnion cell population in the presence of the perturbation with one or more gene signatures or pathways of the amnion cell model or isolated amnion cell population in the absence of the perturbation.

23. The method of claim 15, wherein determining the perturbation-induced change in the one or more gene signatures or pathways comprises performing single cell sequencing.

24. The method of claim 15, wherein the amnion epithelial cell markers comprise GABRP and KRT18, and the amnion mesenchymal cell markers comprise MGP and VIM.

25.-31. (canceled)

32. A method of differentiating stem cells into amnion cells, comprising
culturing a population of induced pluripotent stem (iPS) cells in the presence of one or more agents capable of modulating expression of one or more amnion marker genes in a culture medium under a condition allowing the iPS cells to differentiate into amnion cells, wherein the one or more amnion marker genes is selected from the group consisting of: GABRP, KRT18, IGFBP2, IGFBP5, NPY, FN1, ITGB6, VTCN1, CLDN6, STMN1, TYMS, MAD2L1, CDK1, ZWINT, TK1, CDH1, MGP, DLK1, VIM, IGFL2, POSTN, COL8A2, COL15A1, COL6A1, COL5A1, ADAMTS9, PCLAF, CENPK, CENPM, COL6A1, MALAT1, MRC1, CD36, SRGN, FOLR2, PTPRC, or a combination thereof; and
measuring the expression level of amnion marker genes comprising GABRP, KRT18, MGP, and VIM.

33. The method of claim 32, wherein the one or more agents comprises one or more cytokines, growth factors, hormones, transcription factors, and/or signaling molecules.

34. The method of claim 33, wherein the signaling molecules are selected from the group consisting of: BMP10, BMP3, BTC, MIF, IGF2, PDGFA, AREG, PDGFC, WNT2B, WNT10A, WNT1, WNT10B, WNT5A, WNT7A, WNT4, WNT6, WNT3, BMP7, NOG, PDGFD, WNT9B, INHBB, WNT7B, EGF, MSTN, GDF11, WNT3A, NDP, WNT8B, BMP6, TGFA, GDF9, CHRD, GREM2, MDK, BMP4, WNT2, TGFB2, TGFB3, BMP5, HGF, FST, WNT16, LEFTY1, WNT9A, TGFB1, BMP2, LEFTY2, SPP1, IGF1, HBEGF, PDGFB, WNT11, GDF5, GDF15, GREM1, EPGN, INHBA, and a combination thereof, and/or
wherein the transcription factors are selected from the group consisting of: POU5F1, HAND1, ISL1, ID3, MSX1, EPAS1, ID4, PRRX1, EN1, HOXB6, ID1, DLX5, ID2, MYBL2, TCF4, HOXA10, PPARG, MSX2, TBX3, HES1, TFAP2B, TFAP2A, IRX1, IRX2, MEIS2, GATA6, HAND2, LMCD1, PITX1, PITX2, SOX6, FOSL2, TWIST1, LEF1, ZEB1, TBX2, and a combination thereof.

35. (canceled)

36. The method of claim 32, wherein the one or more agents comprises BMP4.

37.-43. (canceled)

44. The method of claim 15, wherein the one or more amnion epithelial cell markers are selected from the group consisting of: GABRP, KRT18, IGFBP2, IGFBP5, NPY, FN1, ITGB6, VTCN1, CLDN6, STMN1, TYMS, MAD2L1, CDK1, ZWINT, TK1, CDH1, and a combination thereof,
the one or more amnion epithelial stem cell markers are selected from the group consisting of: GABRP, KRT18, IGFBP2, VTCN1, CLDN6, STMN1, TYMS, MAD2L1, CDK1, ZWINT, TK1, CDH1, and a combination thereof,
the one or more amnion mesenchymal cell markers are selected from the group consisting of: MGP, DLK1, VIM, IGFL2, POSTN, COL8A2, COL15A1, COL6A1, COL5A1, ADAMTS9, and a combination thereof,
the one or more amnion mesenchymal stem cell markers are selected from the group consisting of: STMN1, TYMS, MAD2L1, CDK1, MGP, DLK1, VIM, IGFL2, POSTN, PCLAF, CENPK, CENPM, ZWINT, TK1, and a combination thereof,
the one or more fibroblast markers are selected from the group consisting of: COL6A1, COL5A1, ADAMTS9, MALAT1, DLK1, POSTN, and a combination thereof, and/or
the one or more macrophage markers are selected from the group consisting of: MRC1, CD36, SRGN, FOLR2, PTPRC, and a combination thereof.

45. The method of claim 15, wherein the fibroblast markers comprise COL6A1 and COL5A1, and the macrophage markers comprise MRC1 and CD36.

46. The method of claim 15, wherein the amnion cell model or the isolated amnion cell population comprise amnion cells i) obtained from a human amnion sample; ii) obtained from an in vitro generated amnion model, or iii) generated by differentiation of pluripotent or multipotent stem cells.

47. The method of claim 46, wherein the amnion cells are obtained from a first trimester human amnion tissue.

48. The method of claim 15, wherein the amnion cell model or the isolated amnion cell population comprise amnion cells corresponding to human amnion cells in Carnegie stage (CS) 5, 6, 7, 8, 9, 10, 11, 12, 13, 14, 15, 16, 17, 18, 19, 20, 21, 22, or 23.

49. The method of claim 15, wherein the amnion cell model or the isolated amnion cell population comprise engineered amnion cells enriched in transcription factors GATA6, TWIST1 and ZEB1 or enriched in transcription factors TFAP2A and TFAP2B.
